(19) United States
(12) Patent Application Publication
(10) Pub. No.: US 2025/0000586 A1
(43) Pub. Date: Jan. 2, 2025

(54) METHODS AND DEVICES FOR DETERMINING BONE CHARACTERISTICS AND BONE PREPARATION PLANS

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Erik Woodard, Memphis, TN (US); Brett J. Bell, Mendon, UT (US); Forrest A. James, Oakland, TN (US); Branislav Jaramaz, Pittsburgh, PA (US); Constantinos Nikou, Monroeville, PA (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/722,273

(22) PCT Filed: Jan. 6, 2023
(86) PCT No.: PCT/US2023/010268

§ 371 Date: Jun. 20, 2024

Related U.S. Application Data
(60) Provisional application No. 63/297,402, filed on Jan. 7, 2022.

(51) Int. Cl. A61B 34/10; A61B 5/00; A61B 17/16

(52) U.S. Cl. CPC A61B 34/10; A61B 5/6878; A61B 17/1626; A61B 2034/107

(57) ABSTRACT

Trial implant components or inserts (105), bone preparation tools (750), and methods for using same to determine bone characteristics and bone preparation processes for differentially preparing bone regions for cementless implants based on bone characteristics are disclosed. For example, in any preceding or subsequent example, a trial implant component may be configured as a bone property measurement trial implant configured to be inserted within a joint to determine measurement trial information of a bone in forceful contact therewith. In another example, operating characteristics of a bone preparation tool (e.g., voltage, torque, and/or the like) in contact with a patient bone may be measured. The measurement trial information or operating characteristics may be converted to bone characteristic information, such as bone hardness. The bone characteristic information may be used to determine, inter alia, whether a cementless implant may be supported and/or a bone preparation process for an implant.

### CROSS-REFERENCE TO RELATED APPLICATION

This application claims the benefit of U.S. Provisional Patent Application Ser. No. 63/297,402, filed Jan. 7, 2022, and titled “Methods and Devices for Determining Bone Characteristics and Bone Preparation Plans,” the entire contents of which application is hereby incorporated by reference in its entirety.

### TECHNICAL FIELD

The present disclosure is directed to orthopedic surgical methods and devices, and, more specifically, to trial implant devices and bone preparation tools configured to determine bone characteristics, such as bone hardness, and to determining bone preparation plans based on the bone characteristics, for instance, to differentially prepare portions of bony anatomy to receive an implant based on bone hardness values.

### BACKGROUND

In joint replacement surgery, a surgeon typically reshapes one or more bones of the joint and implants a prosthetic component configured to provide the structure and function of the joint. For example, for a knee arthroplasty procedure (such as a total knee arthroplasty (TKA)), the distal end of the femur and the proximal end of the tibia may be resected and reshaped to receive femoral and tibial prosthetic components.

Joint replacement surgeries may use cemented, cementless, or hybrid (relying on both cemented and cementless elements) implant components. In general, cementless (interference-fit or press-fit) implants use friction forces to form an interference fit between the implant component and the prepared bone. Subsequent bone growth facilitates osseointegration to fixate the implant with the bone. Cemented implants are affixed to portions of patient bone via a cement compound.

There are advantages and disadvantages for both cemented implants and cementless implants. For TKA, cemented fixation has been the “gold standard” surgical technique. However, as cementless implants and surgical techniques have improved, preference for cementless implants has increased. In addition, the use of bone cement to attach a prosthetic component onto a prepared bone provides immediate fixation but has various disadvantages that appear over time. For example, physical loads are repeatedly applied to the implant over its life. If bone cement is used to secure a prosthetic component, the bone cement may fatigue and fracture under repeated loading. Such fatigue may lead to degradation of the bone cement integrity, causing the component to become loose, thereby necessitating replacement. Furthermore, there are certain patients where cementless implants may be preferred, such as younger and more active patients.

Cementless joint replacement surgery relies on viable bone to facilitate osseointegration and provide long-term, durable fixation. Bone hardness is a primary measure of bone viability. In general, bone hardness is characterized by resistance to penetration and/or indentation. During a conventional joint replacement procedure, a surgeon may make a visual and/or manual inspection of the bone to determine whether the quality is sufficient to support cementless implants. For example, a surgeon may determine hardness by applying manual pressure (e.g., using a thumb or finger) on the surface of the resected bone. If the pressure causes the bone to deflect, the hardness and quality may be deemed insufficient to support a cementless implant. However, basing a major surgical decision on feel and/or other non-quantitative assessments leads to inaccurate results and poor surgical outcomes.

In addition, cementless implants typically utilize an interference-fit mechanism for coupling the implant to a resected bone surface. The recessions for cementless implants are sized to allow the implant to achieve the interference-fit. More specifically, instead of the bone surface being sized to be exactly complementary to the implant, a degree of interference exists in the fit to provide adequate coupling (for instance, the bone may be prepared to have a larger area than required). In conventional systems, the degree of interference is a standard, pre-set value that is applied consistently across a population of patients. However, various patient-specific factors may affect the fit of the implant. For example, bone quality and/or bone density may affect the firmness of the fit between the implant and the bone. Standard surgical techniques are not capable of efficiently and accurately determining such patient-specific factors and, therefore, do not provide bone preparation techniques for cementless implants that are configured specifically for the bone characteristics of each individual patient.

It is with this in mind that the present disclosure is provided.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Disclosed herein are improved arthroplasty devices and techniques. In any preceding or subsequent example, an arthroplasty device may be in the form of a bone property measurement device or trial implant (“measurement trial”). The measurement trial may be placed in contact with prepared (for instance, resected, cut, and/or the like) bone to be evaluated for one or more properties of the bone. In any preceding or subsequent example, the measurement trial may be configured to evaluate properties of cancellous bone. A non-limiting example of a bone property may include bone hardness and/or other indicators of bone quality for assessing whether the patient anatomy may support a cementless implant. The bone may be positioned or otherwise moved through a range of motion to cause the measurement trial to make certain measurements (for instance, via associated pressure, force, or other sensors) and/or to deform to allow for certain measurements (for instance, a measurement in the change of volume, size, shape, or other characteristics).

In conventional joint replacement procedures, such as a TKA procedure, surgeons generally rely on feel when broaching/punching tibial and femoral bone to assess the bone hardness and whether or not it is sufficient for cementless TKA. If the bone is too soft, cementless TKA may not be the best option for the patient. However, such determinations are highly subjective and prone to error because, among other reasons, they rely on non-quantitative examination methods that are not based in data-driven criteria. In addition, less experienced surgeons may not have the knowledge to make a determination intraoperatively based on manual inspection.

Standard techniques have attempted to overcome these deficiencies through diagnostic imaging, imaging sensors, and/or hardness detectors. However, such methods are challenging to use and require complicated equipment to be used in the operating room, requiring additional resources and time. Hardness detectors, in particular, are not configured to easily measure the pertinent areas of a patient joint, such as the prepared portions of a femur and/or tibia, intramedullary canal, and/or the like.

During a joint replacement procedure, such as a TKA, a surgeon typically uses a trial component or insert that is a temporary implant device that is implanted or placed in association with the prepared (for instance, cut and/or resected) bone of the patient in the same or similar position as the final or permanent implant component in order to examine the patient to determine properties of the final implants, such as size and positioning. In any preceding or subsequent example, a bone property measurement trial implant (“measurement trial”) may be configured to measure one or more physical characteristics of the bone anatomy of a patient. For example, a measurement trial may be configured to determine the hardness of patient bone. In any preceding or subsequent example, the measurement trial may include sensors configured to measure a force or pressure that may be translated to a bone hardness value. In any preceding or subsequent example, the measurement trial may be formed of a deformable material configured to be deformed by patient bone based on the hardness of the patient bone. The amount of deformation may be measured (either directly or indirectly based on a change in fluid-storing characteristics of the measurement trial) to determine the hardness of patient bone.

Measurement trials configured according to any preceding or subsequent example and methods for using measurement trials may provide multiple technological advantages over conventional devices and techniques. In one non-limiting example of a technological advantage, a measurement trial may be configured to measure the hardness of patient bone in a trial form factor that allows for the same or similar trial techniques known to surgeons for determining final implant size and shape to be applied to determining bone hardness. In another non-limiting example of a technological advantage, a hardness determination process using a measurement trial may allow for a comprehensive measurement of multiple areas of patient bone (for instance, resected tibia surface, resected femur surface, intramedullary canal, cuts configured to receive affixation elements (keels, pegs, and/or the like), and/or the like) in an efficient, accurate, and simple process that requires less steps and time than conventional tool-based methods. In a further non-limiting example of a technological advantage, a measurement trial may facilitate a quantitative, data-driven approach to determining bone hardness or other quality metrics than currently employed in traditional surgical techniques. In an additional non-limiting example of a technological advantage, a measurement trial may facilitate the generation of a bone hardness map during an intraoperative procedure indicating the level of hardness of multiple portions of a patient joint. A person of skill in the art would recognize other technological advantages based on the teachings of the present disclosure. Examples are not limited in this context.

In any preceding or subsequent example, a measurement trial may be configured to be inserted into a joint (e.g., knee or hip joint) or portion of a bone or joint (e.g., an acetabular cup) in contact with, or configured to allow for contact with, at least one bone of the joint to determine measurement trial information for the at least one bone. The measurement trial information may be or may be used to determine a bone hardness value. The measurement trial information may be or may include a pressure, a force, deformation information, fluid-storage information, and/or the like. In any preceding or subsequent example, the bone hardness value may be used to determine whether the joint is suitable for cementless, cemented, or hybrid implants. In any preceding or subsequent example, the bone hardness value may be used to determine properties of an implant component, including, without limitation, size, shape, material, and/or the like.

In any preceding or subsequent example, a sensor-based measurement trial may include at least one sensor configured to detect measurement trial information. The at least one sensor may be a pressure sensor and/or a force sensor. The measurement trial information may include a pressure value and/or a force value generated in response to a force of a bone on the measurement trial. The measurement trial information may be used to determine a hardness value associated with the bone.

In any preceding or subsequent example of the sensor-based measurement trial, a logic device may be configured to receive the measurement trial information and generate the hardness value based on the measurement trial information. In any preceding or subsequent example of the sensor-based measurement trial, a logic device may be configured to convert a pressure value or a force value into a hardness value. In any preceding or subsequent example of the sensor-based measurement trial, the logic device may be arranged within the sensor-based measurement trial. In any preceding or subsequent example of the sensor-based measurement trial, the sensor-based measurement trial may include at least one communication element configured to transmit the measurement trial information to a computing device.

In any preceding or subsequent example of the sensor-based measurement trial, the at least one sensor may be arranged within the sensor-based measurement trial. In any preceding or subsequent example of the sensor-based measurement trial, the at least one sensor may be arranged on an external surface of the sensor-based measurement trial. In any preceding or subsequent example of the sensor-based measurement trial, the sensor-based measurement trial may include at least one affixation element and the at least one sensor may be arranged within or on at least one affixation element. In any preceding or subsequent example, the at least one affixation element may include a keel, a peg, or an anchor.

In any preceding or subsequent example of the sensor-based measurement trial, the at least one sensor may include at least one of a MEMS sensor, thin-film pressure sensor, pressure transducer, micro pressure sensor, pressure sensitive film, or a force transducer.

In any preceding or subsequent example, a deformable measurement trial may have at least one portion configured to be deformed by a known amount of force. The deformable measurement trial may be configured to be inserted within a prepared joint and subjected to forces by at least one bone of the joint. At least one deformation may be created on the deformable measurement trial in response to the forces. A reading device may be configured to determine deformation information associated with the deformations. The deformation information may include a number, size, location, depth, and/or the like of the deformations. The deformation information may be used to determine a hardness value associated with the bone or portion of bone that contacted the deformable measurement trial.

In any preceding or subsequent example of the deformable measurement trial, least a portion of the deformable measurement trial may be formed of a material of known compliance, elasticity, modulus, and/or the like. Non-limiting examples, of materials may include rubber, polyphenylsulfone (for example, Radel®), polyetheretherketone (PEEK), ultra-high molecular weight polyethylene (UHMW PE), acetal, polyoxymethylene, polymers, silicone, variations thereof, combinations thereof, and/or the like.

In any preceding or subsequent example of the deformable measurement trial, the reading device may be a laser scanning device, a 3D photogrammetry device, an imaging device.

In any preceding or subsequent example of the deformable measurement trial, the deformable measurement trial may include at least one deformable affixation element configured to generate measurement trial information. In any preceding or subsequent example, the at least one affixation element may include a keel, a peg, or an anchor.

In any preceding or subsequent example, a fluid-based measurement trial may have at least one portion configured to be deformed and a cavity configured to store a fluid arranged therein. The fluid-based measurement trial may be configured to be inserted within a prepared joint and subjected to forces by at least one bone of the joint. The forces may deform at least a portion of the fluid-based measurement trial, thereby causing a change in the fluid-storage characteristics of the cavity.

In any preceding or subsequent example of the fluid-based measurement trial, the cavity may be empty of fluid when initially inserted into the joint and configured to store an initial volume of fluid (for example, at a known pressure or injection pressure). Deformation of the fluid-based measurement trial may cause corresponding deformation of the cavity, for instance, changing the volume of fluid capable of being stored in the cavity (a modified fluid volume) and/or the pressure required to inject the initial volume of fluid into the cavity (a modified fluid pressure). The measurement trial information may be or may include the modified fluid volume and/or the modified fluid pressure. The measurement trial information may be used to determine the bone hardness of the bone that caused the deformations.

In any preceding or subsequent example of the fluid-based measurement trial, the cavity may be storing fluid when initially inserted into the joint at a known pressure. Deformation of the fluid-based measurement trial may cause corresponding deformation of the cavity, for instance, changing the pressure of the fluid stored in the cavity. The measurement trial information may be or may include the pressure of the fluid in the deformed cavity. The measurement trial information may be used to determine the bone hardness of the bone that caused the deformations.

In any preceding or subsequent example, the bone hardness value may be based on the Mohs hardness scale or a specialized hardness index. In any preceding or subsequent example, the measurement trial may be configured for one or more of a resected tibia surface, a resected femur surface, an intramedullary canal, an acetabulum. In any preceding or subsequent example, the measurement trial may include a femoral measurement trial. In any preceding or subsequent example, the measurement trial may include a tibial measurement trial. In any preceding or subsequent example, the measurement trial my include a femoral measurement trial and a tibial measurement trial.

In any preceding or subsequent example, a measurement trial may include an acetabulum measurement trial, for example, in the form of an expandable cup. In any preceding or subsequent example, the expansion of the acetabulum measurement trial may be associated with a bone characteristic, such as bone hardness.

In any preceding or subsequent example, a trial configuration process may be configured to determine the configuration of a measurement trial to optimize hardness measurement for determining whether a patient outcome will be successful for cementless or hybrid implants and/or to determine properties of an implant component, such as size, shape, materials, and/or the like. In any preceding or subsequent example, the trial configuration process may be based on historical patient data indicating implant survivorship. In any preceding or subsequent example, the trial configuration process may be based on mapping portions of the bone where data should be collected to improve implant survivorship.

In any preceding or subsequent example, a bone preparation process may be used to prepare a portion of patient bony anatomy to accept a cementless (or cementless portion of a hybrid) implant based on a bone preparation plan. The cementless implant may be configured to be coupled to at least a portion of the patient bony anatomy via a friction-based fit, such as an interference-fit, press-fit, and/or the like (the terms friction-fit, interference-fit, and press-fit may be used interchangeably in the present disclosure).

The bone preparation process may include determining various bone characteristics of the patient bony anatomy (or portion thereof) indicative of bone quality, such as bone hardness, bone density, bone elasticity, and/or the like. In any preceding or subsequent example, a bone characteristic, such as bone hardness, may be used to determine or predict other values, such as bone density, volume, and/or the like.

A virtual bone characteristic map, grid, or other structure of the patient bony anatomy (for example, a femur or tibia and/or portions thereof for receiving an implant) may be generated that indicates bone characteristic regions (for instance, areas of bone hardness). The bone preparation process may include preparing (for instance, cutting, resecting, planing, burring, and/or the like) portions of the patient bony anatomy based on the bone preparation plan according to the bone characteristic regions. In this manner, a patient may be fitted with an implant using an interference-fit that is individualized for the particular patient bone characteristics, as opposed to using standard patient population and/or manufacturer recommended information according to conventional techniques. In addition, different portions or regions of the patient bony anatomy may be prepared based on the particular bone characteristics of that region.

Cementless implants typically utilize an interference-fit mechanism for coupling the implant to a resected or otherwise prepared bone surface. The recessions for cementless implants are sized to allow the implant to be press-fit onto the bone. Accordingly, instead of the bone surface and the implant being perfectly complementary, a degree of interference exists in the fit to provide adequate coupling. Typically, the degree of interference is a standard, pre-set value based on patient population and/or manufacturer information that is applied across many patients.

Interference-fit fixation is achieved during surgery when the implant component is impacted onto (for example, for a femoral component) or into (for example, for an intramedullary stem) the bone, which is cut slightly larger than the internal dimensions of an implant that fits around the bone (for example, for a femoral component) or smaller than the external dimensions of an implant that fits within the bone (for example, for a intramedullary stem). This size difference between the implant and the bone is an interference fit and is responsible for the compressive stresses acting at the bone-implant interface to hold the implant in place on the patient bony anatomy.

However, various patient-specific factors may affect the interference-fit of the implant. For example, bone quality and/or bone density may affect the firmness of the fit between the implant and the bone. Where the bone is relatively softer or weaker (i.e., lower bone hardness), the fit may be looser with the pre-set interference value, which may lead to instability. Where the bone is relatively harder or stronger (i.e., higher bone hardness), the fit may be tighter with the pre-set interference value, which may lead to difficulty applying the implant to the bone and/or injury or damage in the process. The correct degree of interference balances the stability of the implant with the ease of applying the implant. Accordingly, preceding or subsequent examples may provide bone preparation processes configured to customize the degree of interference in the recession(s) based on the patient bone characteristics, such as quality, hardness, and/or the like.

In any preceding or subsequent example, a bone preparation process may include determining an interference-fit value for a portion of a bone, determining a bone quality characteristic for the portion of the bone, and generating an offset value for the interference-fit value based on the bone quality characteristic.

In any preceding or subsequent example, a surgical method may include preparing the portion of the bone according to the interference-fit value and the offset value. In any preceding or subsequent example, the surgical method may include installing the implant on the bone prepared according to the interference-fit value and the offset value.

In any preceding or subsequent example, the interference-fit value may include a degree of interference between the portion of the bone and an implant component to provide adequate coupling between one of the portion of the bone or the bone and the implant component. In any preceding or subsequent example, the interference-fit value may be an amount of bone of the portion to retain to achieve an adequate interference-fit of an implant component with the portion of the bone.

In any preceding or subsequent example, the bone quality characteristic may include a bone hardness value.

In any preceding or subsequent example, the offset value may include an adjustment to the interference-fit value based on the bone hardness.

In any preceding or subsequent example, the portion of the bone may include a plurality of regions. In any preceding or subsequent example, the bone preparation process may include determining the bone quality characteristic for each of the plurality of regions. In any preceding or subsequent example, the bone preparation process may include determining an offset value for each of the plurality of regions.

In any preceding or subsequent example, the bone quality characteristic may be determined via one or more of physical inspection, a measurement trial, biometric information, diagnostic imaging, bone preparation tool information, or bone analysis preparations.

In any preceding or subsequent example, the bone preparation tool information may include operating characteristics of a bone preparation tool. In any preceding or subsequent example, the operating characteristics may include one or more of resistance, torque, power, voltage, resistance to rotation (for example, a level of resistance to rotation of a rotating object, for instance, applied as torque) and/or amperage. In any preceding or subsequent example, the bone preparation tool may include one or more of a burr or a saw.

In any preceding or subsequent example, a computer-assisted surgical system may include a bone preparation tool and at least one computing device in communication with the bone preparation tool. The at least one computing device may include processing circuitry and a memory coupled to the processing circuitry. The memory may include instructions that, when executed by the processing circuitry, may cause the processing circuitry to: receive operating information of the bone preparation tool during contact of the bone preparation tool with a portion of patient bone, and determine bone characteristic information of the portion of the patient bone based on the operating information.

In any preceding or subsequent example of the computer-assisted surgical system, the bone preparation tool may include at least one of a burring tool, a cutting tool, a saw, a reamer, a broach, or a drill.

In any preceding or subsequent example of the computer-assisted surgical system, the bone preparation tool may include a burring tool.

In any preceding or subsequent example of the computer-assisted surgical system, the bone characteristic information may include bone hardness.

In any preceding or subsequent example of the computer-assisted surgical system, the operating information may include at least one of voltage, amperage, torque, resistance, or rotation speed.

In any preceding or subsequent example of the computer-assisted surgical system, the memory may include instructions that, when executed by the processing circuitry, may cause the processing circuitry to: access an interference-fit value for a cementless implant component for the portion of the patient bone, and determine at least one implant component property of the cementless implant based on the bone characteristic information and the interference-fit value. In any preceding or subsequent example of the computer-assisted surgical system, the at least one implant component property may include a size of the cementless implant. In any preceding or subsequent example of the computer-assisted surgical system, the at least one implant component property may include a shape of the cementless implant.

In any preceding or subsequent example of the computer-assisted surgical system, the memory may include instructions that, when executed by the processing circuitry, may cause the processing circuitry to: access an interference-fit value for a cementless implant component for the portion of the patient bone, and generate an offset value based on the bone characteristic information, the offset value comprising an adjustment to the interference-fit value.

In any preceding or subsequent example of the computer-assisted surgical system, the bone characteristic information may include a hardness value for each of a plurality of different regions of the portion of patient bone.

In any preceding or subsequent example of the computer-assisted surgical system, the memory may include instructions that, when executed by the processing circuitry, may cause the processing circuitry to generate an interference-fit value map to visually represent interference-fit values and corresponding offsets for the plurality of different regions of the portion of patient bone.

In any preceding or subsequent example of the computer-assisted surgical system, the memory may include instructions that, when executed by the processing circuitry, may cause the processing circuitry to generate a bone preparation plan for a cementless implant component based, at least in part, on the bone characteristic information. In any preceding or subsequent example, generating a bone preparation plan may include updating, revising, or otherwise modifying a prior or existing bone preparation plan.

In any preceding or subsequent example of the computer-assisted surgical system, the portion of patient bone may include at least one of a tibia or a femur.

In any preceding or subsequent example, a computer-implemented method may include, via at least one processor of at least one computing device, receiving operating information of a bone preparation tool during contact of the bone preparation tool with a portion of patient bone; accessing operational data associated with the bone preparation tool with at least one object of known bone characteristic information; and converting the operating information to patient bone characteristic information for the portion of patient bone based on the operational data.

In any preceding or subsequent example of the computer-implemented method, the bone preparation tool may include at least one of a burring tool, a cutting tool, a saw, a reamer, a broach, or a drill.

In any preceding or subsequent example of the computer-implemented method, the bone preparation tool may include a burring tool.

In any preceding or subsequent example of the computer-implemented method, the bone characteristic information may include bone hardness.

In any preceding or subsequent example of the computer-implemented method, the operating information may include at least one of voltage, amperage, torque, resistance, or rotation speed.

In any preceding or subsequent example, the computer-implemented method may include accessing an interference-fit value for a cementless implant component for the portion of the patient bone, and determining at least one implant component property of the cementless implant based on the bone characteristic information and the interference-fit value. In any preceding or subsequent example of the computer-implemented method, the at least one implant component property may include a size of the cementless implant. In any preceding or subsequent example of the computer-implemented method, the at least one implant component property may include a shape of the cementless implant.

In any preceding or subsequent example of the computer-implemented method, the method may further include accessing an interference-fit value for a cementless implant component for the portion of the patient bone, and generating an offset value based on the bone characteristic information, the offset value comprising an adjustment to the interference-fit value.

In any preceding or subsequent example of the computer-implemented method, the bone characteristic information may include a hardness value for each of a plurality of different regions of the portion of patient bone.

In any preceding or subsequent example of the computer-implemented method, the method may further include generating an interference-fit value map to visually represent interference-fit values and corresponding offsets for plurality of different regions of the portion of patient bone.

In any preceding or subsequent example of the computer-implemented method, the method may further include generating a bone preparation plan for a cementless implant component based on the bone characteristic information. In any preceding or subsequent example, generating a bone preparation plan may include updating, revising, or otherwise modifying an existing bone preparation plan.

In any preceding or subsequent example of the computer-implemented method, the portion of patient bone may include at least one of a tibia or a femur.

In any preceding or subsequent example, a sensor-based measurement trial implant may include a main body configured to be inserted within a joint of a patient in contact with at least one bone of the joint; and at least one pressure sensor arranged to measure force information of a force imparted on the main body via movement of the at least one bone against the main body, the force information configured to correspond to bone characteristic information of the at least one bone.

In any preceding or subsequent example of the sensor-based measurement trial implant, the bone characteristic information may include bone hardness.

In any preceding or subsequent example of the sensor-based measurement trial implant, the at least one pressure sensor may be configured to measure force-displacement of at least a portion of the at least one bone.

In any preceding or subsequent example of the sensor-based measurement trial implant, the at least one pressure sensor may include a plurality of pressure sensors, each of the plurality of pressure sensors may be configured to determine bone characteristic information of a different portion of the at least one bone.

In any preceding or subsequent example of the sensor-based measurement trial implant, the sensor-based measurement trial implant may further include a stem configured to be arranged within an intramedullary canal of the at least one bone, at least one of the plurality of pressure sensors may be associated with the stem to measure force information of the intramedullary canal.

In any preceding or subsequent example, a deformable measurement trial implant may include a main body configured to be inserted within a joint of a patient in contact with at least one bone of the joint; and at least one deformable portion configured to be deformed to generate at least one deformation responsive to a force imparted on the main body via movement of the at least one bone against the main body, the at least one deformation may be configured to be read by a reading device to determine deformation information configured to correspond to bone characteristic information of the at least one bone.

In any preceding or subsequent example of the deformable measurement trial implant, the bone characteristic information may include bone hardness.

In any preceding or subsequent example of the deformable measurement trial implant, the deformation information may include at least one of a size, a location, a depth of the at least one deformation.

In any preceding or subsequent example of the deformable measurement trial implant, the reading device may include at least one of a laser scanning device, a three-dimensional photogrammetry device, or an imaging device.

In any preceding or subsequent example of the deformable measurement trial implant, the at least one deformable portion may include at least one deformable affixation element in the form of at least one of a keel, a peg, or an anchor.

In any preceding or subsequent example, a fluid-based measurement trial implant may include a main body configured to be inserted within a joint of a patient in contact with at least one bone of the joint; and at least one cavity having at least one fluid-storage characteristic and configured to receive a fluid, the at least one fluid-storage characteristic of the at least one cavity is modified responsive to a force imparted on the main body via movement of the at least one bone against the main body, the at least one fluid-storage characteristic corresponds to bone characteristic information of the at least one bone.

In any preceding or subsequent example of the deformable measurement trial implant, the bone characteristic information may include bone hardness.

In any preceding or subsequent example of the deformable measurement trial implant, the at least one fluid-storage characteristic may include a volume of fluid capable of being stored in the at least one cavity.

In any preceding or subsequent example of the deformable measurement trial implant, the at least one fluid-storage characteristic may include a pressure of a fluid being stored in the at least one cavity.

In any preceding or subsequent example of the deformable measurement trial implant, the main body may be configured to be deformed to cause corresponding deformation of the at least one cavity, thereby modifying the at least one fluid-storage characteristic of the at least one cavity.

In any preceding or subsequent example, a computer-assisted surgical system may include a measurement trial component configured to be inserted within a joint of a patient in contact with at least one bone of the joint, the measurement trial component configured to generate measurement trial information responsive to a force imparted on the measurement trial component via movement of the at least one bone against the measurement trial component; and at least one computing device that may include processing circuitry and a memory coupled to the processing circuitry. The memory may include instructions that, when executed by the processing circuitry, may cause the processing circuitry to receive the measurement trial information of the measurement trial component and determine bone characteristic information of the at least one bone based on the measurement trial information.

In any preceding or subsequent example of the computer-assisted surgical system, the bone characteristic information may include bone hardness.

In any preceding or subsequent example of the computer-assisted surgical system, the memory may include instructions that, when executed by the processing circuitry, may cause the processing circuitry to access an interference-fit value for a cementless implant component for the portion of the patient bone, and generate an offset value based on the bone characteristic information, the offset value comprising an adjustment to the interference-fit value.

In any preceding or subsequent example of the computer-assisted surgical system, the bone characteristic information may include a hardness value for each of a plurality of different regions of the portion of patient bone.

In any preceding or subsequent example of the computer-assisted surgical system, the memory may include instructions that, when executed by the processing circuitry, may cause the processing circuitry to generate an interference-fit value map to visually represent interference-fit values and corresponding offsets for the plurality of different regions of the portion of patient bone.

In any preceding or subsequent example of the computer-assisted surgical system, the memory may include instructions that, when executed by the processing circuitry, may cause the processing circuitry to generate a bone preparation plan for a cementless implant component based on the bone characteristic information. In any preceding or subsequent example, generating a bone preparation plan may include updating, revising, or otherwise modifying an existing bone preparation plan.

In any preceding or subsequent example of the computer-assisted surgical system, the portion of patient bone may include at least one of a tibia or a femur.

In any preceding or subsequent example of the computer-assisted surgical system, the measurement trial component may include a sensor-based measurement trial implant that may include a main body configured to be inserted within a joint of a patient in contact with at least one bone of the joint; and at least one pressure sensor arranged to measure force information of a force imparted on the main body via movement of the at least one bone against the main body, the force information may be configured to correspond to bone characteristic information of the at least one bone

In any preceding or subsequent example of the computer-assisted surgical system, the measurement trial component may include a deformable measurement trial implant that may include a main body configured to be inserted within a joint of a patient in contact with at least one bone of the joint; and at least one deformable portion configured to be deformed to generate at least one deformation responsive to a force imparted on the main body via movement of the at least one bone against the main body, the deformation may be configured to be read by a reading device to determine deformation information configured to correspond to bone characteristic information of the at least one bone.

In any preceding or subsequent example of the computer-assisted surgical system, the measurement trial component may include a fluid-based measurement trial implant that may include a main body configured to be inserted within a joint of a patient in contact with at least one bone of the joint; and at least one cavity having at least one fluid-storage characteristic and configured to receive a fluid, the at least one fluid-storage characteristic of the at least one cavity is modified responsive to a force imparted on the main body via movement of the at least one bone against the main body, the at least one fluid-storage characteristic corresponds to bone characteristic information of the at least one bone.

In any preceding or subsequent example, the portion of patient bone may include at least one of a portion of a knee joint, a portion of a hip joint, a portion of a shoulder joint, a tibia, a femur, a humerus, a glenoid, an acetabulum, a pelvis.

A bone preparation process according to any preceding or subsequent example may provide multiple technological advantages over conventional devices and techniques. In one non-limiting example of a technological advantage, a bone preparation process may operate to adjust the bone preparation based on the patient bone quality, which facilitates an improved, patient-specific interference-fit, reducing risk and improving quality-of-life for the patient. In an additional non-limiting example of a technological advantage, a bone preparation process may provide for differential interference-fit values for different regions of a bone (for example, to accommodate different bone quality characteristics that may be exhibited throughout a bone surface, intramedullary canal, and/or the like).

In another non-limiting example of a technological advantage, a bone preparation process may provide for the determination and use of a patient-specific offset value, rather than a standard or generic value. This provides much better stability for the implant and ease of applying the implant (i.e., low risk of damage). Bone preparation processes according to any preceding or subsequent example may also permit the use of bone quality data most relevant to the patient at the time of the operation, for instance, patient-specific, in real time, and evaluated local to the cut surface. Therefore, bone preparation processes may allow for dynamic variation of the offset value across a cut surface to provide an optimal and patient-specific interference-fit over an entire bone surface.

Further features and advantages of at least some of the examples of the described technology, as well as the structure and operation of various examples of the described technology, are described in detail below with reference to the accompanying drawings.

### BRIEF DESCRIPTION OF THE DRAWINGS

Examples of the disclosed device will now be described, with reference to the accompanying drawings, in which:

FIG. 1 illustrates an exemplary trial implant system that can be utilized for a knee replacement procedure in accordance with one or more features of the present disclosure;

FIG. 2A illustrates a side perspective view of a bone property measurement trial implant in accordance with one or more features of the present disclosure;

FIGS. 2B and 2C illustrate a side perspective view and a top-down view of a bone property measurement trial implant for a femur in accordance with one or more features of the present disclosure;

FIGS. 2D and 2E illustrate a side perspective view and a top-down view of a bone property measurement trial implant for a tibia in accordance with one or more features of the present disclosure;

FIG. 3 illustrates a side perspective view of an internal sensor bone property measurement trial implant in accordance with one or more features of the present disclosure;

FIG. 4 illustrates a side perspective view of an external sensor bone property measurement trial implant in accordance with one or more features of the present disclosure;

FIG. 5 illustrates a property assessment process using a deformable bone property measurement trial implant in accordance with one or more features of the present disclosure;

FIG. 6 illustrates a property assessment process using a fluid-based bone property measurement trial implant in accordance with one or more features of the present disclosure;

FIG. 7 is a diagram illustrating an environment for operating a system for planning and performing a joint replacement surgery in accordance with one or more features of the present disclosure;

FIG. 8 is a block diagram depicting a system for performing a surgery planning process in accordance with one or more features of the present disclosure; and

FIG. 9A illustrates an exemplary bone hardness map in accordance with one or more features of the present disclosure.

FIG. 9B illustrates an exemplary bone region indicator in accordance with one or more features of the present disclosure;

FIG. 9C illustrates exemplary bone characteristic information determined via dual-energy X-ray absorptiometry (DEXA) in accordance with one or more features of the present disclosure;

FIG. 9D illustrates an exemplary bone analysis preparation in accordance with one or more features of the present disclosure;

FIG. 10A illustrates a first block diagram of an exemplary interference fit in accordance with one or more features of the present disclosure;

FIG. 10B illustrates a second block diagram of an exemplary interference fit in accordance with one or more features of the present disclosure;

FIG. 11 illustrates an exemplary bone preparation process workflow for determining bone characteristic information based on bone preparation tool operating information in accordance with one or more features of the present disclosure;

FIG. 12 illustrates an exemplary bone preparation process workflow in accordance with one or more features of the present disclosure; and

FIG. 13 illustrates an exemplary interference-fit map in accordance with one or more features of the present disclosure.

It should be understood that the drawings are not necessarily to scale and that the disclosed examples are sometimes illustrated diagrammatically and in partial views. In certain instances, details which are not necessary for an understanding of the disclosed methods and devices or which render other details difficult to perceive may have been omitted. It should be further understood that this disclosure is not limited to the particular preceding or subsequent examples illustrated herein. In the drawings, like numbers refer to like elements throughout unless otherwise noted.

### DETAILED DESCRIPTION

Various features of arthroplasty devices, techniques for using arthroplasty devices, bone preparation tools, and bone preparation processes will now be described more fully hereinafter with reference to the accompanying drawings, in which one or more features of the arthroplasty devices, techniques for using arthroplasty devices, and bone preparation processes will be shown and described. It should be appreciated that the various features or the like described hereinafter may be used independently of, or in combination, with each other. It will be appreciated that an implant device, system, component, process, technique, and/or portion thereof as disclosed herein may be embodied in many different forms and should not be construed as being limited to the preceding or subsequent examples set forth herein. Rather, these preceding or subsequent examples are provided so that this disclosure will convey certain or features of the implant devices to those skilled in the art.

Disclosed herein are implant devices in the form of a bone property measurement trial implant (“measurement trial”) including one or more features for enabling, inter alia, measurement of one or more properties of a bone. Disclosed herein are measurement bone preparation tools configured to measure one or more properties of a bone during operation, for instance, while in contact with patient bone. A non-limiting example of a bone property may be or may include bone hardness. Illustrative and non-restrictive examples of anatomical structures may be or may include a knee joint, a hip joint, a shoulder joint, a tibia, a femur, a humerus, a glenoid, a pelvis, an acetabulum, cut portions thereof, and/or resected portions thereof.

In general, bone hardness is a measure of the ability of a bone to resist indentation, flexion, or similar localized plastic deformation in response to a force. Bone hardness is an indicator of bone quality. The hardness of a bone may be measured based on a scale or index, such as the Mohs hardness scale, or a specialized hardness index. As a non-limiting example, bone is generally considered to be rated as a 5 out of 10 on the Mohs hardness scale.

Although bone hardness is used as an example bone property in the present disclosure, preceding or subsequent examples are not so limited. For example, a bone property may include any property that may indicate whether a joint, bone, or other anatomical structure may support a cementless implant device. In another example, a bone property may include any property that may be used to determine a property of an implant component (cemented, cementless, hybrid, and/or the like), such as a size, shape, material, and/or the like of the implant component. A knee joint, a tibia, a femur, and/or arthroplasty knee surgery (total knee arthroplasty (TKA), unicompartmental knee arthroplasty (UKA), and/or the like) may be used in examples in the present disclosure. However, preceding or subsequent examples are not so limited. For instance, measurement trials, measurement bone preparation tools, and surgical techniques described in the present disclosure may be applied to other anatomical structures and/or joints (for instance, hip joints).

Although knee anatomy, TKA, and other knee-related examples are used in the present disclosure, preceding or subsequent examples are not so limited. More specifically, other orthopedic surgical techniques, implants, and/or patient anatomy may be used according to any preceding or subsequent examples. For example, preceding or subsequent examples may be used with hip orthopedic surgical techniques, including, without limitation sizing the implant component interference with cup impaction techniques.

Although cementless implants and determining whether a patient anatomy can support a cementless implant are used in examples in the present disclosure, examples are not so limited. In particular, processes, devices, and/or the like according to any preceding or subsequent example may be applied to determining properties of any type of implant component (cementless, cemented, hybrid, and/or other implant types), including, without limitation, size, shape, materials, type, and/or the like.

A trial component or insert is a temporary implant device that is implanted or placed in the patient in the same or similar position as the final or permanent implant component in order to examine the patient to determine properties of the final implants, such as size and positioning. For example, in a TKA, the initial surgical steps comprise resecting and shaping the distal end of the femur and the proximal end of the tibia to receive the femoral and tibial prostheses. Trial femoral and/or tibial prostheses are then temporarily implanted into the patient in their respective positions. The joint is then examined, including during extension and flexion of the joint, to assess the performance of the trial components. Different types and/or sizes of trials may be used to determine the optimal final implant components. The surgeon may make additional cuts and/or reshape the distal end of the femur and/or the proximal end of the tibia (i.e., bone preparation techniques) based on the performance of the trial to achieve an optimal fit of the final implant components.

Conventional trial components may be used to determine physical characteristics of the final implant components, such as the size, and/or whether further bone preparations are required. However, existing trial components are not capable of determining physical properties of the bone. More specifically, conventional trial components are not capable of directly determining the hardness of the bone. Accordingly, in any preceding or subsequent example, measurement trials may be configured to provide the conventional functions of a trial component (i.e., determining type and size of final component and/or bone shaping) as well as determining measurement trial information. In any preceding or subsequent example, the measurement trial information may include deformation information, pressure or force information, fluid volume information, fluid pressure information, and/or the like. The measurement trial information may be used to determine physical properties of the bone, such as bone hardness.

In any preceding or subsequent example, a measurement trial may be a sensor-based measurement trial that includes one or more sensors, such as a force or pressure sensor. The sensors may be arranged on the inside and/or outside of the sensor-based measurement trial. The sensor-based measurement trial may be implanted in the patient in contact with bone. Movement of the bone (for instance, flexion and extension of a knee joint) may cause the bone to press on the sensor-based measurement trial. The force resulting from the bone pressing on the sensor-based measurement trial may be detected by the sensors to generate measurement trial information, for instance, in the form of force or pressure measurements. The measurement trial information may be translated into a measurement of the hardness of the examined bone area.

In any preceding or subsequent example, the measurement trial may be in the form of a deformable measurement trial. The deformable measurement trial may be formed of a material configured to deform a known amount in response to a force. The deformable measurement trial may be implanted in the patient in contact with bone. Movement of the bone may cause the bone to press on the deformable measurement trial. The force resulting from the bone pressing on the deformable measurement trial may be cause the deformable measurement trial to become deformed. The deformable measurement trial in the deformed state may be measured to determine measurement trial information in the form of deformation information (for instance, number of deformations, locations of deformations, size/area of deformations, depth of deformations, and/or the like). The deformation information may be translated into a measurement of the hardness of the examined bone area.

In any preceding or subsequent example, the measurement trial may be in the form of a fluid-based measurement trial. The fluid-based measurement trial may be formed of a material configured to deform in response to a force. A cavity configured to receive and store a known volume of a fluid may be arranged within the fluid-based measurement trial. In any preceding or subsequent example, the volume of fluid may be stored at a known pressure. The fluid-based measurement trial may be implanted in the patient in contact with bone. Movement of the bone may cause the bone to press on the fluid-based measurement trial. The force resulting from the bone pressing on the fluid-based deformable measurement trial may be cause the fluid-based measurement trial to become deformed and, as a result, the cavity may also be deformed. Deformation of the cavity may change the volume of fluid that may be stored in the cavity and/or change the pressure of the fluid in the cavity (and any fluid paths). The volume and/or pressure of the fluid that is stored in the cavity may be detected as measurement trial information, which may be translated into a measurement of the hardness of the examined bone area.

Accordingly, any preceding or subsequent example may provide measurement trials configured to determine measurement trial information via direct contact with one or more bones of a joint. The measurement trial information may be used to precisely and accurately determine bone characteristics, including bone hardness.

Conventional bone preparation tools may be used for bone preparation techniques, such as modifying patient bone to accept an implant, for instance, via sawing, cutting, burring, and/or the like. However, existing bone preparation tools are not capable of determining physical properties of the bone during operation. For instance, conventional bone preparation tools are not capable of directly determining the hardness of the bone, for instance, during normal operation of the tool (for example, a conventional burring tool is not capable of determining the hardness of a portion of bone during active burring of the bone). Accordingly, in any preceding or subsequent example, measurement bone preparation tools may be configured to provide the conventional functions of a bone preparation tool (e.g., cutting, sawing, burring, and/or the like) as well as measuring certain operating characteristics or information that may be used to determine bone characteristics, such as bone hardness, while performing conventional functions.

A measurement trial and/or measurement bone preparation tool according to any preceding or subsequent examples may be used during a surgical procedure to determine bone hardness to, among other things, provide an assessment of the quality of a joint, bone, or portion thereof. A bone hardness determination made using a measurement trial and/or measurement bone preparation tool according to any preceding or subsequent examples may be used to perform various evaluations, including, without limitation, whether the patient anatomy is suitable for a cementless (or hybrid) implant system, implant characteristics (for instance, size, shape, material, offsets, and/or the like). In this manner, a surgeon may perform an assessment of bone quality that does not rely on inaccurate and subjective tests employed using conventional devices and techniques.

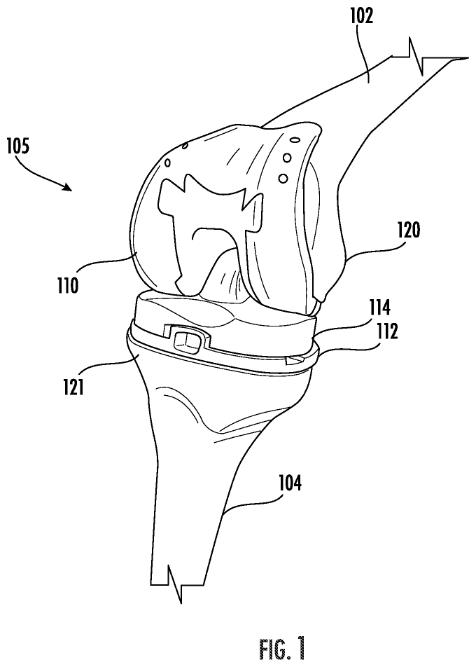

FIG. 1 illustrates an exemplary trial implant system that can be utilized for a knee replacement procedure in accordance with one or more features of the present disclosure. As shown in FIG. 1, a distal end 120 of a femur 102 and a proximal end of a tibia 104 may be prepared by a surgeon to receive implant components. A trial implant system 105 may include a femoral trial 110 and a tibial or baseplate trial 114. Femoral trial 110 may be positioned on distal end 120 of femur 102, and tibial trial 112 may be placed on proximal end 121 of tibia 104. An insert trial (articular insert trial) 114 may be installed onto the tibial trial. In any preceding or subsequent example, trial implant system 105 may be the same or substantially similar to a trial implant system used in combination with one or more of the Journey™ and/or Journey II™ systems manufactured and sold by Smith & Nephew, Inc. of Cordova, Tennessee, United States.

During the surgical procedure, trial implant system 105 may be evaluated for proper sizing of the final femur and tibial implant components. Different sized and/or shaped trial components may be used to determine the optimal final implant components. In addition, a trial range of motion may be performed to assess performance characteristics of trial implant system 105, including alignment, laxity, and balance. Distal end 120 of femur 102 and/or proximal end 121 of tibia 104 may be cut and/or reshaped based on the performance assessment to provide an optimal fit of the final implant components.

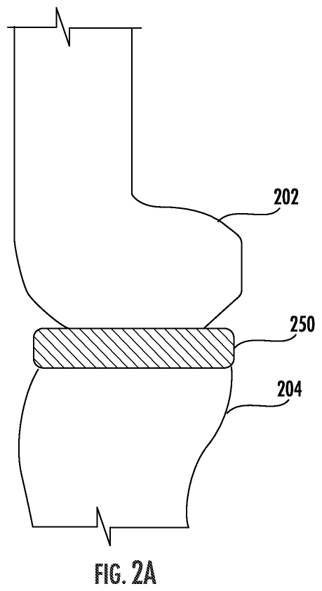

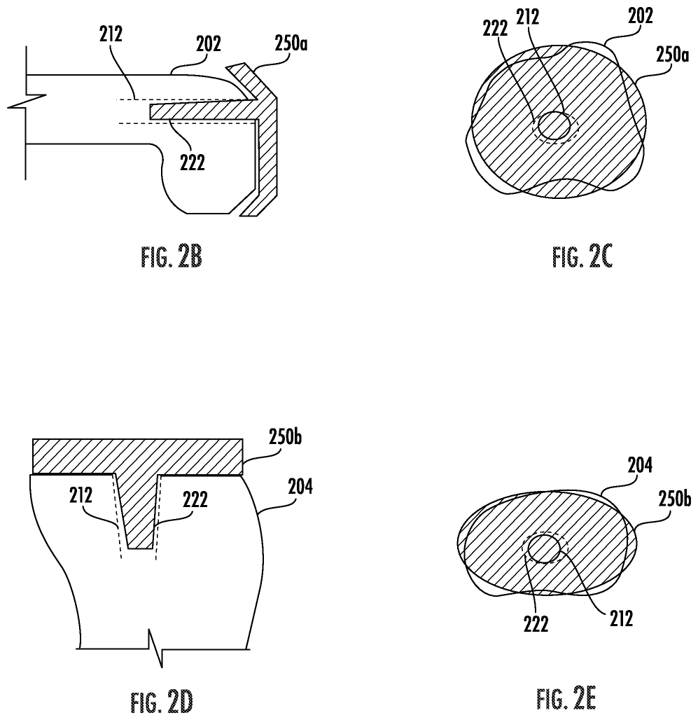

Although trial implant system 105 is depicted as including the three components of a femoral trial 102, a tibial or baseplate trial 104, and an insert trial 114, preceding or subsequent examples are not so limited, as more, fewer, and/or additional components may be used. In addition, more, fewer, and/or additional components may be used for different examinations of the patient anatomy. For example, a measurement trial (see, for example, FIG. 2A) may be used to evaluate bone characteristics, such as bone hardness, to determine whether cemented, cementless, or hybrid components may be used and/or properties (of cemented or cementless) implant components, such as size, shape, materials, and/or the like. Subsequently, the components of trial implant system 105 may be used to determine the characteristics of the final implant components (e.g., size, shape, and/or the like) and/or whether further cuts and/or shaping of the bone is necessary.

FIG. 2A illustrates a side perspective view of a measurement trial in accordance with one or more features of the present disclosure. As shown in FIG. 2A, a measurement trial 250 may be placed between prepared ends of a femur 202 and tibia 204 of a knee joint. For example, the bone may be prepared with a punch, bur, broach, saw, and/or the like. After bone preparation, measurement trial 250 may be impacted into the prepared cavity. In any preceding or subsequent example, measurement trial 250 may be a single trial component that operates without a traditional femoral trial and/or tibial trial in order to directly contact the bone. In any preceding or subsequent example, measurement trial 250 may be configured to determine properties of femur 202, of tibia 204, and/or both femur 202 and tibia 204. As described in further detail in the present disclosure, in any preceding or subsequent example, measurement trial 250 may include various elements, such as a stem, keel, projections, and/or the like to promote implantation and/or to correspond to elements of the final implant components.

In any preceding or subsequent example, measurement trial 250 may include a stem, wedge, or other structure that corresponds to a stem of an implant, such as an intramedullary stem of a revision TKA implant. The depth of insertion of the stem may be an indicator of bone quality, such as bone hardness (i.e., the further the stem is able to be inserted, the lower the bone hardness). The stem may be graduated or otherwise include indications of insertion depth. The insertion depth may be used, for instance, in a pass/fail capacity in which an insertion depth over a threshold depth may indicate that the bone is too soft to support cementless fixation. In this manner, measurement trials 250 may be used to provide objective feedback to a surgeon whether to use cemented stems or press-fit stems.

FIGS. 2B and 2C illustrate a side perspective view and a top-down view of a measurement trial for a femur in accordance with one or more features of the present disclosure. As shown in FIGS. 2B and 2C, in any preceding or subsequent example, measurement trial (or “femoral measurement trial”) 250a may be configured for implantation on femur 202. For example, measurement trial 250a may include a stem 222 configured to be installed within an intramedullary canal 212 of femur 202.

FIGS. 2D and 2E illustrate a side perspective view and a top-down view of a measurement trial for a tibia in accordance with one or more features of the present disclosure. As shown in FIGS. 2D and 2E, in any preceding or subsequent example, measurement trial (or “tibial measurement trial”) 250b may be configured for implantation on tibia 204. For example, measurement trial 250b may include a stem 222 configured to be installed within an intramedullary canal 212 of tibia 204.

In any preceding or subsequent example, only one measurement trial 250 may be used. In any preceding or subsequent example, measurement trial 250 may not be particularized for either femur 202 or tibia 204 (for example, a “neutral measurement trial”). In any preceding or subsequent example, only one of a femoral measurement trial 250a or tibial measurement trial 250b may be used. In any preceding or subsequent example, both femoral measurement trial 250a and tibial measurement trial 250b may be used. In any preceding or subsequent example, one of a femoral measurement trial 250a or tibial measurement trial 250b may be used in combination with a neutral measurement trial.

In any preceding or subsequent example, a bone assessment process may be configured to assess a property of at least one bone of a joint for determining at least one final implant component for a joint replacement surgery. In any preceding or subsequent example, bone assessment process may be or may include a hardness assessment process configured to use measurement trial 250 determine the hardness of at least a portion of at least one bone of the joint. For example, hardness assessment process may be performed to determine a hardness of at least a portion of femur 202 and/or tibia 204, for instance, prepared surfaces of femur 202 and/or tibia 204. The determined hardness may be used to evaluate whether cemented, cementless, or hybrid final implant components may be used for a joint replacement surgery and/or properties of a cemented, cementless, or hybrid implant component, such as size, shape, type, materials, and/or the like.

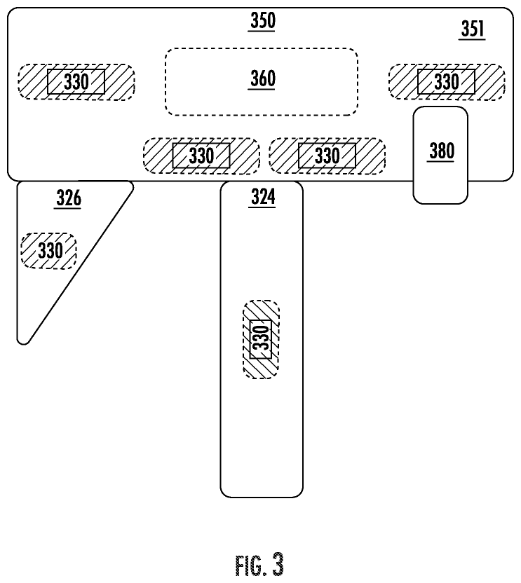

FIG. 3 illustrates a side perspective view of an internal sensor measurement trial in accordance with one or more features of the present disclosure. As shown in FIG. 3, a measurement trial 350 may have one or more pressure or force sensors 330 arranged internally within measurement trial 350. Sensors 330 may include various types of sensors, including, without limitation, MEMS sensors, thin-film pressure sensors, pressure transducers, micro pressure sensors, pressure sensitive film, a force transducer, and/or the like. A non-limiting example of a sensor or sensor system may include a pressure mapping sensor provided by Tekscan, Inc. of South Boston, Massachusetts, United States.

In any preceding or subsequent example, measurement trial 350 may be configured as a neutral measurement trial, a femoral measurement trial, or a tibial measurement trial. In any preceding or subsequent example, measurement trial 350 may include various fixation elements, including, without limitation, a stem, a keel, a fin, a projection, a groove, a peg, and/or the like. For example, measurement trial 350 includes a stem 324 and a keel 326. Pressure sensors 330 may be arranged within a main body 351 of measurement trial 350 and/or within the fixation elements, such as stem 324 and keel 326.

Measurement trial 350 may be configured such that forces imparted on measurement trial 350 by a bone may be detected by one or more of pressure sensors 330. For example, measurement trial 350 may be placed or implanted on a distal end of a femur. The knee joint may be flexed such that the distal end of the femur (and/or proximal end of the tibia) imparts a force on measurement trial 350. The force may be measured or otherwise detected by pressure sensors 330. In any preceding or subsequent example, at least a portion of measurement trial 350 may be deformed by the force, and this deformation may be detected by pressure sensors 330. For example, a mapping process may determine critical areas of the bone to be measured to assess hardness. The mapping process may be performed via direct examination and/or based on historical data. For example, direct examination and/or historical data may indicate that the hardness of regions X, Y, and Z are most important in determining the outcome of a cemented, cementless, and/or hybrid joint replacement and/or determining properties of a cemented, cementless, and/or hybrid implant component. Accordingly, measurement trial 350 may be configured to determine the hardness at regions X, Y, and Z, for instance, by forming corresponding portions of measurement trial 350 with a known elasticity modulus that may facilitate detection of the hardness of the bone via pressure sensors 330.

In any preceding or subsequent example, sensors 330 may be configured to determine force-displacement of at least a portion of the bone. In any preceding or subsequent example, at least a portion of sensors 330 may be or may include force sensors, torque sensors, displacement transducers, and/or the like configured to determine force-displacement. Accordingly, in any preceding or subsequent example, data may be captured of force displacement over a known area of the bone, including, for instance, a force-displacement curve. The force-displacement information may be used to determine bone characteristics, such as bone hardness.

In any preceding or subsequent example, at least a portion of measurement trial 350 may be formed of a material of known compliance, elasticity, modulus, and/or the like. Non-limiting examples, of materials may include rubber, polyphenylsulfone (for example, Radel®), polyetheretherketone (PEEK), ultra-high molecular weight polyethylene (UHMW PE), acetal, polyoxymethylene, polymers, silicone, variations thereof, combinations thereof, and/or the like.

In any preceding or subsequent example, measurement trial 350 may not deform and pressure sensors 330 may be configured and/or arranged to detect the force without the deformation of measurement trial 350.

Measurement trial 350 and/or sensors 330 may be configured such that a detected force or pressure measured by sensors 330 may be used to derive, calculate, or otherwise determine a hardness of the bone. For example, measurement trial 350 may be inserted into the cavity between a prepared femur and tibia. The joint may be positioned at one or more flexion or extension angles and the pressure detected via at least one of pressure sensors 330. The detected pressure may be used to determine a hardness or other quality indicator of the prepared femur and/or tibia. For example, a detected pressure of X may indicate a hardness of Y. In another example, a detected pressure of X at a flexion angle of Z may indicate a hardness of Y. In a further example, pressure and hardness relationships may be used to estimate bone hardness. A non-limiting example of a pressure-hardness relationship may include the following Meyer's Law: P=kdⁿ, where P is the applied pressure, k is the resistance of material to initial penetration, n is Meyer's index (a measure of the effect of deformation on the hardness of the material), and d is the diameter of indentation. In another example, experimental data from known material properties may be used to relate pressure measurements, for instance, from measurement trial sensors to traditional hardness measurement techniques such as micro-indentation. The relative bone hardness may also be estimated by comparing the deformation of the trial in bone to the deformation in a reference material, such as polymethylmethacrylate (PMMA).

In any preceding or subsequent example, a set or subset of sensors 330 may be used to determine properties of the bones of a joint. For instance, a pressure sensor 330 in stem 324 may be used to determine a hardness of an intramedullary canal. In another instance, a pressure sensor 330 in keel 326 may be used to determine a hardness of a cut section of bone. In a further instance, one of sensors 330 may indicate a hardness of a portion of a prepared tibial surface, and another of sensors 330 may indicate a hardness of a portion of a prepared femoral surface.

In any preceding or subsequent example, the pressure or force readings from a combination of sensors 330 may be used to determine a hardness value. In any preceding or subsequent example, the pressure determinations may be used to determine a hardness value on a hardness scale, such as the Mohs hardness scale. In any preceding or subsequent example, the pressure determinations may be used to determine a specialized hardness index. Examples are not limited in this context.

In any preceding or subsequent example, pressure sensors 330 may be configured to communicate with a logic device, such as a computing device, to provide pressure readings for use in determining hardness information (see, for example, FIGS. 7 and 8). In any preceding or subsequent example, the logic device may be or may be a part of a computer assisted surgery (CAS) system (see, for example, FIG. 7). In any preceding or subsequent example, a control device 360 may be arranged within measurement trial 350. In any preceding or subsequent example, control device 360 may be communicatively coupled to pressure sensors 330. Control device 360 may be configured to control various operational features of pressure sensors 330 and/or communicate with a logic device to provide pressure readings for use in determining hardness information. The communication between pressure sensors 330, control device 360, and/or a logic device may be via various wired and/or wireless protocols.

In any preceding or subsequent example, a measurement trial may include one or more cams, screws, locking screws, fasteners, and/or the like configured to modify the position, orientation, location, or other implantation characteristic of the measurement trial. For example, cams or similar mechanisms may be used to simulate different interference levels for a cementless interference-fit, friction-fit, and/or the like. Referring to FIG. 3, measurement trial 350 includes cam 380. Measurement trial 350 may be implanted with cam disengaged 380 under a first set of conditions, such as an interference fit. Sensors 330 may be used to collect information, such as forces, etc. imparted on measurement trial 350 in the first set of conditions. Cam 380 may be engaged, for example, by rotating, etc., to engage cam 380 with a surface of a bone to push on measurement trial 350, for example, to simulate a second set of conditions, such as an increased interference fit between measurement trial 350 and the bone. Sensors 330 may then measure a second set of information for the measurement trial in the second set of conditions.

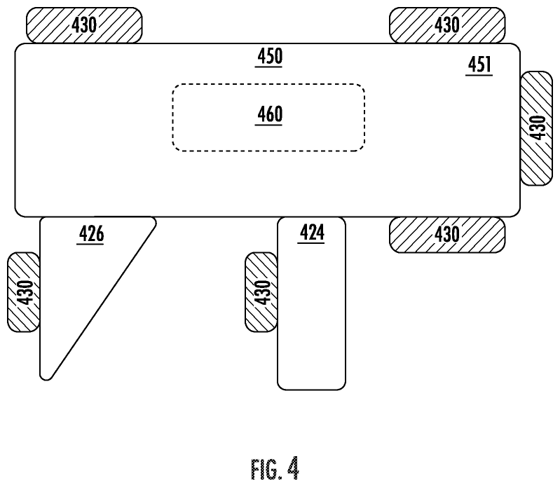

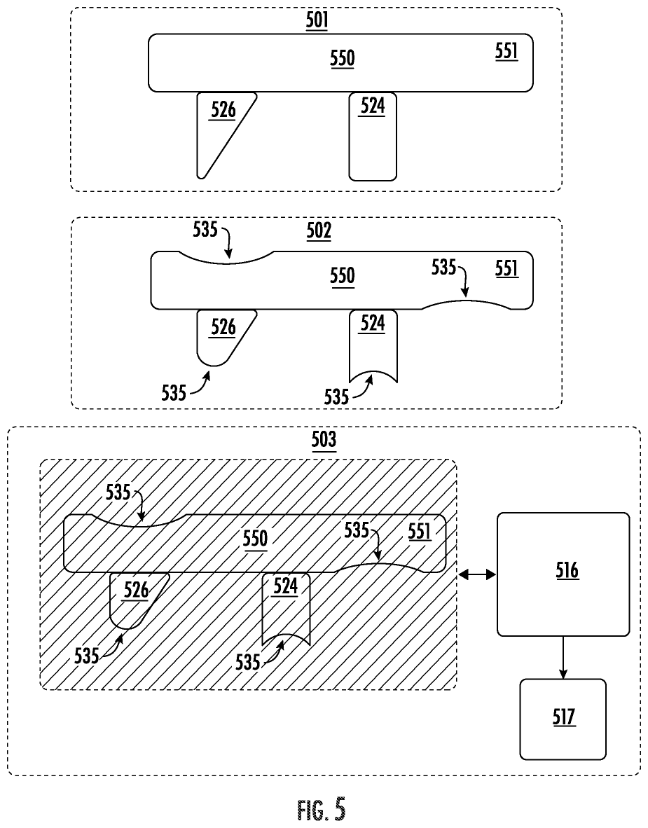

FIG. 4 illustrates a side perspective view of an external sensor measurement trial implant in accordance with one or more features of the present disclosure. As shown in FIG. 4, a measurement trial 450 may include pressure or force sensors 430 arranged on an outside surface of measurement trial 450. In any preceding or subsequent example, measurement trial 450 may include fixation elements, such as stem 424 and/or keel 426. Pressure sensors 430 may be arranged on a main body 351 of measurement trial 350 and/or on fixation elements, such as a stem 424 and a keel 426. In any preceding or subsequent example, a control device 460 may be arranged within measurement trial 450. In any preceding or subsequent example, control device 460 may be communicatively coupled to pressure sensors 330 and/or a logic device (not shown, see FIGS. 7 and 8). In any preceding or subsequent example, the pressure or force readings from one or more of sensors 430 may be used to determine a hardness value for at least a portion of a bone of a joint.

In any preceding or subsequent example, a measurement trial may include a combination of internal sensors (for example, FIG. 3) and external sensors (for example, FIG. 4).

FIG. 5 illustrates a property assessment process using a deformable measurement trial in accordance with one or more features of the present disclosure. As shown in FIG. 5, a first step 501 of a property assessment process may include providing a deformable measurement trial 550 for insertion within a cavity of a prepared knee joint.

Measurement trial 550 may include a main body 551 and one or more fixation elements, such as a stem 524 and/or a keel 526. Measurement trial 550 may be formed of a deformable material with known elasticity or similar properties. The deformation of measurement trial 550 may hold its shape after removal of the force to allow for measurement of the deformation. This deformation information may be used to determine a hardness of the associated portion of bone that imparted the force. In this manner, an amount of deformation may be used to determine a corresponding force imparted on measurement trial 550. Non-limiting examples of materials may include polyphenylsulfone, polyethylene, polyurethane, polymers, silicone, latex, polyols, diisocyanates, methylene diphenyl diisocyanate, toluene diisocyanate, foam, variations thereof, combinations thereof, and/or the like. In any preceding or subsequent example, measurement trial 550 may be less rigid than an implant. Accordingly, measurement trial 550 may compress the associated portion of the bone less than the actual installed implant, including with no or substantially no plastic deformation.

At step 502, portions of the knee joint may contact measurement trial 550. For example, the knee joint may be subjected to flexion, extension, or other ranges of motion. The movement of the knee joint may cause the bones of the joint (for instance, a prepared end of a femur) to contact and impart a force on measurement trial 550. For example, the knee joint may be positioned into a known flexion angle. Deformations 535 may be formed in measurement trial 550, such as indentations, grooves, concave surfaces, and/or the like. Deformations 535 may be permanent or semi-permanent. In any preceding or subsequent example, deformations 535 may be plastic and not elastic (for instance, deformations 535 may retain or substantially retain their shape after removal), for example, the same or similar to a PE foam material (even if other material are used).

At step 503, measurement trial 550 may be removed from the joint. A measurement device 516 may measure properties of deformations 535 to generate deformation information 517. Non-limiting examples of measurement device 516 may include a laser scanning device, a 3D photogrammetry device, an imaging device (for example, to generate images that may be analyzed by a computing device), and/or the like. Deformation information 517 may include information associated with deformations 535, including, without limitation, size, area, volume, depth, shape, location, and/or the like. Deformation information 517 may be used to derive, calculate, or otherwise determine the hardness of the bone or bones under examination. For example, the depth (or other characteristic) of a deformation 535 (for instance, at a particular location on measurement trial 550) may be used to determine a corresponding force which was sufficient to generate the particular deformation 535. This force may be used to determine the hardness of the bone that imparted the force.

In one non-limiting example, a depth of a deformation 535 of X (for instance, at region A) may indicate a hardness of Y. In another non-limiting example, a hardness assessment process may determine a total, average, mean, maximum, minimum, and/or other calculation of a property of deformations 535, which may be used to determine a hardness value. For instance, the total depth of deformations 535 (for example, by adding up the depths of all deformations 535) on a tibial side of measurement device 550 may be used to determine a hardness of the resected surface of the tibia. Examples are not limited in this context.

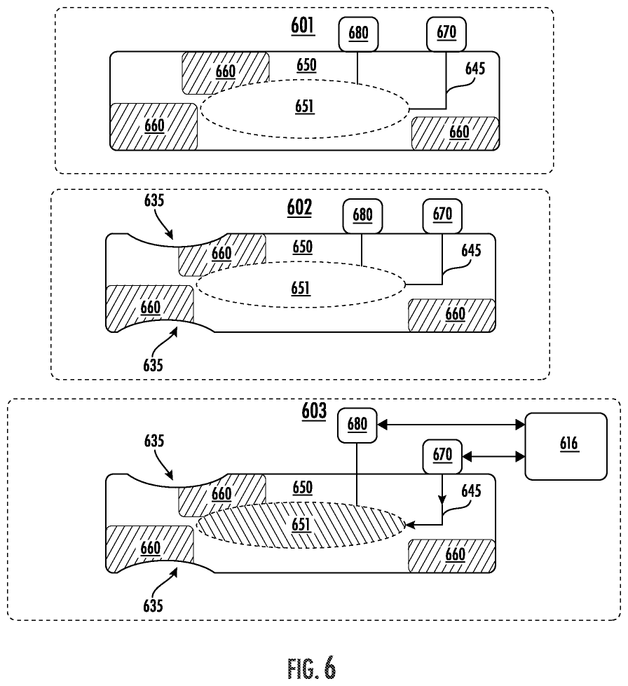

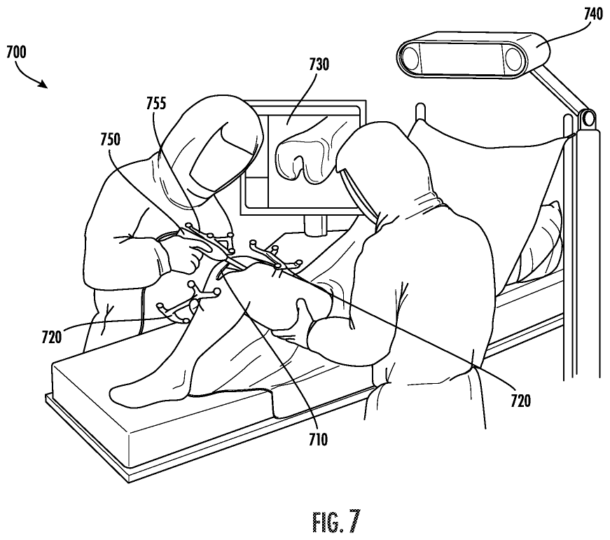

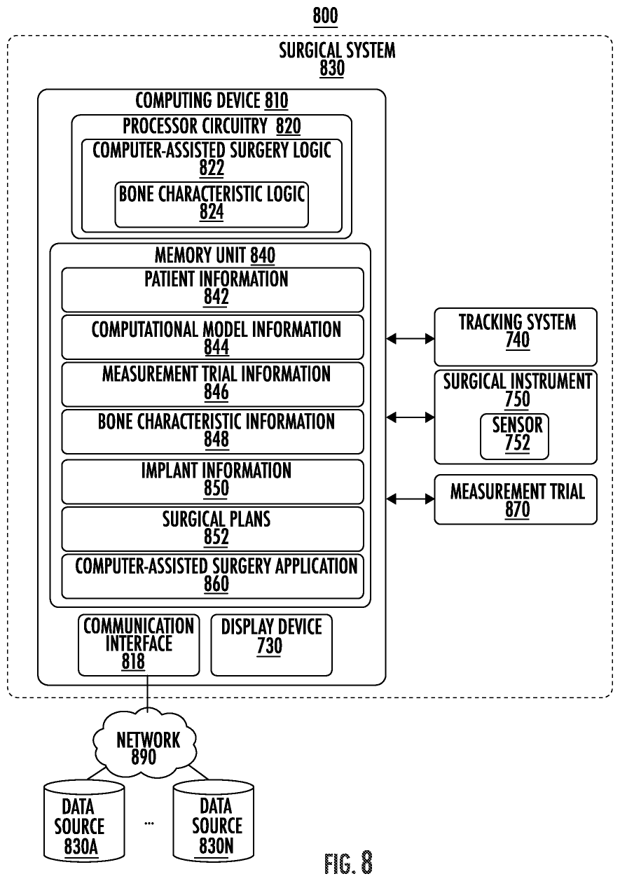

FIG. 6 illustrates a property assessment process using a fluid-based bone property measurement trial implant in accordance with one or more features of the present disclosure. As shown in FIG. 6, a first step 601 of a property assessment process may include providing a fluid-based measurement trial 650 for insertion within a cavity of a prepared knee joint.

Measurement trial 650 may include a main body and one or more fixation elements, such as a stem and/or a keel (not shown). Measurement trial 650 may be formed of a deformable material. In any preceding or subsequent example, the deformation of measurement trial 650 may hold its shape after removal of the force to allow for measurement of the deformation.

In any preceding or subsequent example, measurement trial 650 may include at least one fluid cavity 651. In any preceding or subsequent example, cavity 651 may be fluidically coupled to a fluid source or reservoir 670 via a fluid path 650. Although cavity 651 is depicted as being coupled to fluid source 650 in steps 601 and 602, cavity 651 may be connected to fluid source for fewer steps, for instance, only step 603. Cavity 651 may be configured to receive and store a fluid, including, without limitation, a liquid or gas. Non-limiting examples of the fluid may include water, saline, a saline solution, an oil, a liquid polymer, an inert gas, variations thereof, combinations thereof, and/or the like.

At step 602, portions of the knee joint may contact measurement trial 650. For example, the knee joint may be subjected to flexion, extension, or other ranges of motion. The movement of the knee joint may cause the bones of the joint (for instance, a prepared end of a femur and/or a prepared end of a tibia) to contact and impart a force on measurement trial 650. For example, the knee joint may be positioned into a known flexion angle. Deformations 635 may be formed in measurement trial 650, such as indentations, grooves, concave surfaces, and/or the like.

In any preceding or subsequent example, measurement trial 650 may include at least one deformable zone 660 configured to be susceptible to deformation. In any preceding or subsequent example, deformable zones 660 may be arranged at critical locations, for example, that correspond with critical interface locations between measurement trial 650 and/or components thereof (for instance, affixation components) and the bone(s) of the joint.

In a volume-based process example, cavity 651 may be empty (or substantially empty) or contain a known amount of fluid during step 602 such that cavity 651 may hold a known volume of the fluid (the “initial cavity volume”). Deformation of measurement trial 650 may cause a corresponding deformation or other change in cavity 651. The deformation of cavity 651 may change the volume of fluid that may be held by cavity 651 (the “modified cavity volume”). In general, if cavity 651 is deformed, the modified cavity volume may be less than the initial cavity volume. If there is no deformation of cavity 651, then the modified cavity volume will equal the initial cavity volume.

The fluid may be forced into cavity 651 from fluid source 670 (for example, a fluid-filled syringe) into cavity 651. The injection of the fluid may occur at a known and/or constant pressure. The volume of the injected fluid may be measured, for example, via a measurement device 616 or manually (for instance, reading a fluid level of a syringe) to determine the modified cavity volume. The modified cavity volume is essentially a measure of how much measurement trial 650 was deformed by the contacting bones of the joint. A hardness determination may be calculated based on the modified cavity volume. For example, a modified cavity volume that indicates a volume reduction of cavity 651 of X %, X volume units (for instance, milliliters), or another difference calculation may indicate a bone hardness rating of Y.

In a pressure-based process example, cavity 651 may contain a volume of fluid at a known pressure at step 602 (“initial cavity pressure”) (for instance, a first or baseline point on a pressure curve, such as a stress-strain curve). In any preceding or subsequent example, cavity 651 may be hermetically sealed. Deformation of measurement trial 650 may cause corresponding deformation of cavity 651, thereby changing the pressure of the fluid stored in cavity. A pressure sensor 680 (either internal or external to measurement trial 650) may be configured to measure the pressure of the fluid in deformed cavity 651 (“modified cavity pressure”). In general, if cavity 651 is deformed, the modified cavity pressure may be greater than the initial cavity pressure. If there is no deformation of cavity 651, then the modified cavity pressure will equal the initial cavity pressure.

One or more modified cavity pressures may be used to determine a pressure curve or stress-strain curve. In any preceding or subsequent example, multiple cavities 651 or pressure points may be used to provide localized information (for instance, a first cavity for a first region, a second cavity for a second region, and so on). In any preceding or subsequent example, pressure sensor(s) 680 (or other types of sensors) may be configured to determine force-displacement of at least a portion of the bone. For example, a known quantity of fluid may be injected into cavity 651 while measuring pressure, which may provide a force/displacement curve over a known surface area. The force/displacement curve may be used to determine bone characteristics, such as bone hardness.

A hardness determination may be calculated based on the modified cavity pressure. For example, a modified cavity pressure of X or an increase of Z may indicate a bone hardness rating of Y.

In a pressure-to-volume process example, cavity 651 may be filled with a fluid at step 603. The pressure-to-volume process may determine the filling pressure of injected fluid required to reach a predetermined volume. Deformation of cavity 651 may cause the filling pressure to be increased in order for cavity 651 to receive the predetermined volume of fluid. In any preceding or subsequent example, a hardness determination may be calculated based on the filling pressure. For example, a filling pressure (or an increase in the filling pressure of a non-deformed cavity 651) of X may indicate a bone hardness rating of Y.

Although cavity 651 is shown as being arranged within a main body of measurement trial 650, examples are not so limited. For example, one or more cavities may be arranged in other elements of measurement trial 650, such as a stem, a keel, an anchor, deformable zones, and/or the like. In another example, an element, such as a keel or deformable zone, may expand or be inflated via the injection of the fluid (for instance, to a known pressure). The pressure of the fluid and/or the volume of the fluid in the keel (or another element) may be used to determine bone hardness of corresponding bone structures.

Accordingly, in any preceding or subsequent example, bone hardness may be determined via a measurement device based on one or more of fluid displacement (based on a volume of fluid) and/or pressure (for instance, pressure on a syringe or other device injecting the fluid).

Although measurement trials 350, 450, 550, and 650 of FIGS. 3-6 are formed and described in reference to a knee joint, examples are not so limited. For example, measurement trials 350, 450, 550, and 650 and any of the functional elements and/or features (e.g., sensors, deformable materials, and/or the like) may be configured for different portions of patient anatomy. For instance, measurement trials 350, 450, 550, and 650 may be formed to measure bone characteristics of a hip joint, such as a cup-shaped measurement trial for an acetabulum. For example, one or more of measurement trials 350, 450, 550, and 650 may be configured as an expandable cup to measure the hardness of an acetabulum.

FIG. 7 is a diagram illustrating an environment for operating a system for planning and performing a joint replacement surgery in accordance with one or more features of the present disclosure. As shown in FIG. 7, a system 700 may be configured to perform an arthroplasty surgical procedure using a robotic system. In any preceding or subsequent example, system 700 may be or may include an image-free (for instance, CT-less) system. In any preceding or subsequent example, system 700 may be or may include an image-based system based on diagnostic image data. In any preceding or subsequent example, system 700 may operate using a combination of image-free and image-based processes. Examples are not limited in this context.

System 700 may include a surgical cutting tool 750 (a bone preparation tool or measurement bone preparation tool according to any previous or subsequent example) with an associated optical tracking frame 755 (also referred to as tracking array 755), graphical user interface 730, an optical tracking system 740, and patient tracking frames 720 (also referred to as tracking arrays 720). The illustration also includes an incision 710 through which, for example, an arthroplasty surgery may be performed. In an example, the illustrated robotic surgical system 700 depicts a hand-held computer-controlled surgical robotic system, for instance, the same or similar to the Navio® Surgical System or CORI® surgical system from Smith & Nephew, Inc. of Cordova, Tennessee, United States. System 700 may use an optical tracking system 740, or other type of tracking system, coupled to a robotic controller to track and control a hand-held surgical instrument. For example, optical tracking system 740 tracks tracking array 755 coupled to surgical tool 750 and tracking arrays 720 coupled to the patient to track locations of the instrument relative to the target bone (e.g., femur and/or tibia for knee procedures).

In any preceding or subsequent example, surgical cutting tool 750 may be configured to implement contoured, 3D, concave, convex, slanted, angled, beveled, ribbed, ridged, grooved, or otherwise non-flat or non-planar cuts or resections to patient bone, including surfaces with cavities, recesses, channels, slots, and/or the like.

FIG. 8 is a block diagram depicting a system for planning and/or performing a surgical procedure in accordance with one or more features of the present disclosure. A non-limiting example of a surgical procedure may include an orthopedic or arthroplasty procedure, for instance, a TKA or UKA procedure.

As shown in FIG. 8, operating environment 800 may include a surgical system 830. In any preceding or subsequent example, surgical system 830 may include a computing device 810 communicatively coupled to a network 890 via a communication interface (for instance, a transceiver) 818. Computing device 810 may be or may include one or more logic devices, including, without limitation, a server computer, a client computing device, a personal computer (PC), a workstation, a laptop, a notebook computer, a smart phone, a tablet computing device, and/or the like. Although a single computing device 810 is depicted in FIG. 8, examples are not so limited, as surgical system 830 may include and/or operably communicate with, and surgery planning processes according to any preceding or subsequent examples may be performed using, a plurality of computing devices 810. In addition, components of computing device 810 depicted in FIG. 8 may be arranged within a plurality of different computing devices. Examples are not limited in this context.

In any preceding or subsequent example, surgical system 830 may include computing device 810 operating, for example, as a control system, a tracking system 740, and/or a surgical instrument 750. Optionally, in any preceding or subsequent example, surgical system 830 may also include or may be operatively coupled to a display device 130 and/or a one or more data sources (for instance, databases) 830a-n. In any preceding or subsequent example, display device 130 and/or databases 830a-n may be used to provide information associated with a measurement trial 870 and/or other features of a surgical procedure, such as navigation and control of surgical instrument 750, which may include navigation and control of a cutting tool, a point probe, or other tools/instruments, that may be used during an arthroplasty procedure, such as an orthopedic (or similar) prosthetic implant revision surgery.

In any preceding or subsequent example, communication interface 818 may facilitate communication between control system 810 and external systems and devices, including trial implant device 850. Communication interface 818 may include both wired and wireless communication interfaces, such as Ethernet, IEEE 802.11 wireless, or Bluetooth, among others. As illustrated in FIG. 7, in this example, the primary external systems connected via the communication interface 818 may include tracking system 740 and surgical instrument 750.

Computing device (or control system) 810 may include a processor circuitry 820 that may include and/or may access various logics for performing processes according to any preceding or subsequent examples, for example, a surgical planning process. For instance, processor circuitry 820 may include and/or may access a computer-assisted surgery logic 822 and/or a bone characteristic logic 824. Processing circuitry 820, computer-assisted surgery logic 822, bone characteristic logic 824, and/or portions thereof may be implemented in hardware, software, or a combination thereof. As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution, examples of which are provided by the exemplary computing architecture 900. For example, a logic, circuitry, or a module may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a System-on-A-Chip (SoC), memory units, logic gates, registers, semiconductor device, chips, microchips, chip sets, software components, programs, applications, firmware, software modules, computer code, a control loop, a computational model or application, an AI model or application, an ML model or application, a proportional-integral-derivative (PID) controller, FG circuitry, variations thereof, combinations of any of the foregoing, and/or the like.

Although computer-assisted surgery logic 822 is depicted as being within processor circuitry 820 and bone characteristic logic 824 is depicted as being within computer-assisted surgery logic 822 in FIG. 1, examples are not so limited. For example, computer-assisted surgery logic 822, bone characteristic logic 824, and/or any component thereof may be located within an accelerator, a processor core, an interface, an individual processor die, a memory, a storage device, a data store, a database, implemented entirely or partially as a software application (for instance, a computer-assisted surgery application 860), and/or the like.

Memory unit 840 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In addition, memory unit 840 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid state drive (SSD), and/or the like.

Memory unit 840 may store various types of information and/or applications for performing features and processes according to any preceding or subsequent examples, for instance, a surgical planning process according to any preceding or subsequent examples. For example, memory unit 840 may store patient information 842, computational model information 844, measurement trial information 846, bone characteristic information 848, surgical plans 852, and/or computer-assisted surgery application 860. In any preceding or subsequent example, all or some of the information depicted as being stored in memory unit 840 may be, in whole or in part, stored in data sources 830a-n and accessible to computing device 810.

In any preceding or subsequent example, patient information 842 may include information for a patient undergoing a surgical procedure being performed via surgical system. Patient information 842 may include information associated with a specific patient undergoing the surgical procedure, such as personal information (for instance, name, address, and/or the like), physical characteristics (for instance, height, weight, and/or the like), medical information (for instance, health history, health record identifiers, procedure information, and/or the like), and/or any other type of information that may be associated with a patient.

In any preceding or subsequent example, patient information 842 may include information associated with a population of patients. For example, the population of patients may include individuals that may be used to determine measurement trial configurations. For example, patient information 842 may indicate areas of bone that are optimal for determining successful patient outcomes, for example, of cementless or hybrid implant procedures. In another example, patient information 842 may indicate bone quality factors that are optimal and/or minimum requirements for successful patient outcomes, for example, of cementless or hybrid implant procedures.

In a further example, patient information 842 may include information associating measurement trial information (for instance, pressures for sensor-based measurement trials, deformation characteristics for deformable measurement trials, fluid volumes and/or pressures for fluid-based measurement trials) with bone hardness ratings, indices, or other values. For instance, real-world and/or virtual patients with known bone hardness values may be tested using real-world and/or virtual measurement trials in order to calculate, translate, extrapolate, convert, or otherwise determine bone hardness values from measurement trial information.

In any preceding or subsequent example, computational models 844, such as machine learning (ML), neural network (NN), and/or other artificial intelligence (AI) models may be trained to determine configurations of measurement trials in order to determine bone hardness based on patient information 842 and/or other information. For example, the placement of sensors 330 of measurement trial 350 may be determined via a computational model 844 to optimally determine the hardness of critical bone portions for evaluating a successful cementless or hybrid implant patient candidate and/or properties of cemented, cementless, and/or hybrid implant components, such as size, shape, type, materials, and/or the like. A computational model 844 may be trained on patient information 842 to determine which portions of the bony anatomy may be crucial for determining bone quality. In another example, computational models 844 may be trained to determine the bone quality or hardness values required for a successful cementless or hybrid implant patient candidate.

In any preceding or subsequent example, computational models 844 may be trained based on information associated with a population of patients with the same or similar surgical procedures, implants, anatomy, and/or the like. For example, a NN may be trained to receive input in the form of bone hardness for one or more areas of a joint and whether the surgery was successful or not (e.g., whether there was an implant failure, pain, performance issues and/or the like) and generate one of a measurement trial configuration (for instance, specifying measurement regions) and/or minimum patient hardness values (for instance, for bone region A (resected tibial surface), hardness should be a minimum of X, for bone region B (femoral intramedullary canal), hardness should be a minimum of Y, and so on).

Measurement trial information 846 may include information received from and/or generated by measurement trial 870 (for instance, raw sensor data) and/or a device reading information from measurement trial 870 (for instance, a laser scanner, an imaging device, and/or the like). Measurement trial information 846 may be used to generate bone characteristic information 848. In any preceding or subsequent example, bone characteristic information 848 may be or may include bone quality information, bone hardness information, bone density information, and/or the like. For example, a pressure value from a pressure sensor of measurement trial 870 may be received by computing device 810. Bone characteristic logic 824, alone or in combination with computer-assisted surgery application, may evaluate the pressure value to determine a corresponding hardness value.

In any preceding or subsequent example, measurement trial information 846 may be used in combination with information associated with implant survivorship, complications, and/or the like to implement long term tracking of implant success factors based on quantitative hardness data.

In any preceding or subsequent example, bone characteristic logic 824 may be configured to determine a hardness mapping of one or more bones of a joint. For example, bone characteristic logic 824 may receive measurement trial information from measurement trial 870 and may determine the locations or regions of measurement trial that were the source of the measurement trial information.

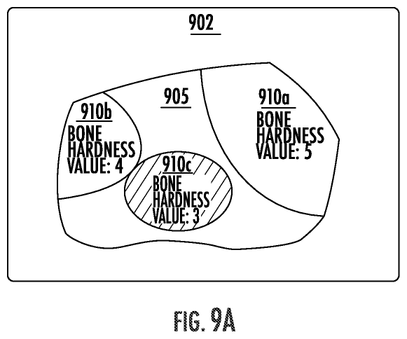

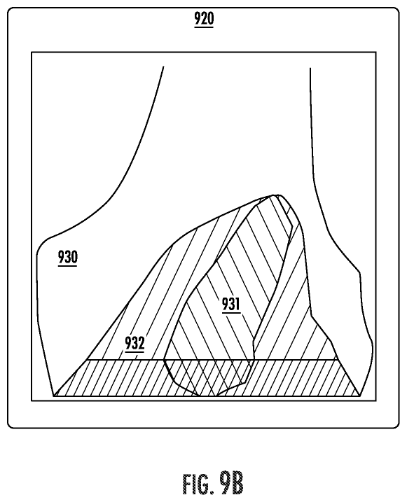

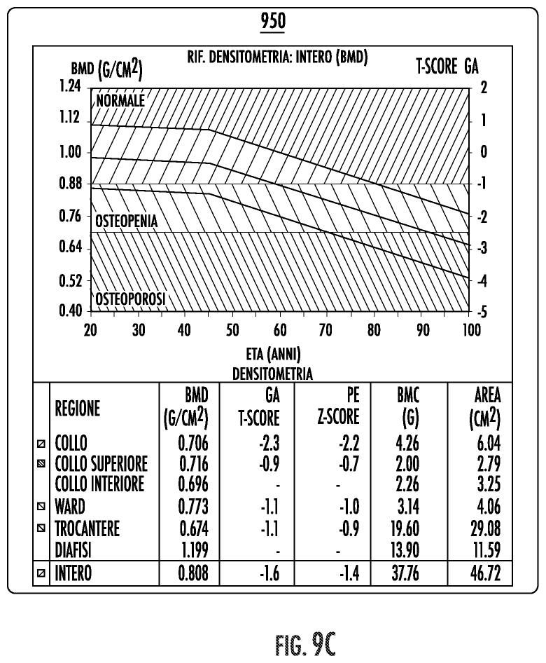

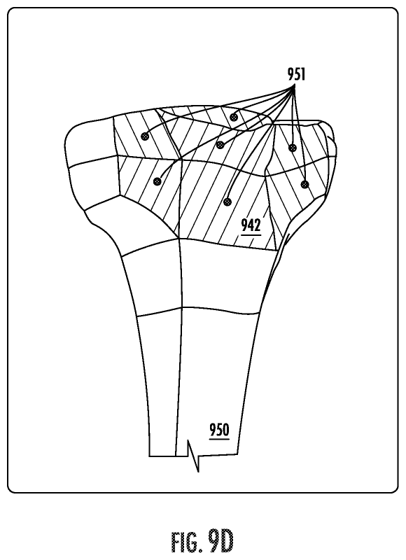

FIG. 9A illustrates an exemplary bone hardness map in accordance with one or more features of the present disclosure. As shown in FIG. 9A, a hardness mapping window 902 (for instance, displayed via display device 730) may present a model 905 or image of a portion of a joint, such as the resected surface of a tibia. One or more regions 910A-N where hardness was determined via a measurement trial may be overlaid on the model 905 along with associated bone characteristic information, such as a bone hardness value. The bone characteristic information may be differentially displayed depending on the value, such as highlighting regions that have a hardness below a threshold (for instance, region 910c) or require attention.

In any preceding or subsequent example, implant information 850 may include information associated with an implant being used in a surgical plan 852. Non-limiting examples of implant information 850 may include dimensions, shape, size, installation information, interference-fit information, interference-fit tolerances, offsets, manufacturer information, and/or the like. In general, implant information 850 may include any information about an implant that may be used by a surgeon to install the implant.

In any preceding or subsequent example, a surgical plan 852 may include instructions, method, steps, workflows, and/or the like for performing a surgical procedure. In any preceding or subsequent example, surgical system 830 may develop a surgical plan 852 based on bone characteristic information 848. For example, surgical plan 852 may include cementless, cemented, or hybrid components depending on bone characteristic information 848. In another example, surgical plan 852 may specify implant component properties, such as size, shape, type, materials, and/or the like based, at least in part, on bone characteristic information 848. Surgical plans 852 may provide a recommended optimal implant size, implant position, implant orientation, implant parameters, and/or the like based on among other things, bone characteristic information 848. In any preceding or subsequent example, developing, generating, processing, or otherwise interacting with a bone preparation plan may include updating, revising, or otherwise modifying an existing bone preparation plan.

Surgical plans 852 may be administered, in whole or in part, manually by a surgeon, automatically (for instance, computer- or robot-assisted) via computer-controlled surgical instruments 750, and/or combinations thereof. Examples are not limited in this context. In any preceding or subsequent example, surgical plans 852 may be or may include a bone preparation plan configured according to any preceding or subsequent examples. For example, a surgical plan 852 may include a bone preparation plan to prepare portions of the bone for an interference-fit. In any preceding or subsequent example, a surgical plan 852 may be or may include an interference-fit map (see, for example, FIG. 13) indicating interference-fit information, such as offset values, interference-fit values, and/or the like for different regions of patient anatomy.

In any preceding or subsequent example, computer-assisted surgery application 860 may be or may include a software application that includes and/or operates in combination with computer-assisted surgery logic 822 and/or bone characteristic logic 824 to perform features of surgical system 830 described in the present disclosure.

In any preceding or subsequent example, surgical instrument 750 may be a measurement bone preparation tool. In any preceding or subsequent example, surgical instrument 750 may be wholly or partially computer-controlled. In any preceding or subsequent example, surgical instrument 750 may be manually controlled (for instance, by a surgeon). In any preceding or subsequent example, surgical instrument 750 may be associated with navigation elements. In any preceding or subsequent example, surgical instrument 750 may not be associated with navigation elements. Non-limiting examples of surgical instrument 750 may include a saw, a burr or burring device, a drill or drilling device, a reamer, a broach, a cutting device, impactor, and/or any other type of tool, instrument, device, and/or the like for modifying bone.

In any preceding or subsequent example, surgical instrument 750 may include one or more sensors 752. In any preceding or subsequent example, sensor 752 may be or may include circuitry, logic, monitor, transducer, wire or wiring, signaling device, and/or other element capable of detecting an operational function of surgical instrument. In any preceding or subsequent example, sensor 752 may determine bone preparation tool information associated with operating characteristics of surgical instrument 750. Non-limiting examples of operating characteristics may include one or more of resistance, torque, power, voltage, amperage, resistance to rotation (for example, a level of resistance to rotation of a rotating object, for instance, applied as torque) and/or amperage. In any preceding or subsequent example, the bone preparation tool may include one or more of a burr or a saw.

In any preceding or subsequent example, bone characteristic information 848 may include information for converting bone preparation tool information to bone characteristic information 848. For example, CAS application 860 may be configured to receive bone preparation tool information, such as a voltage, when surgical instrument 750 is in contact with patient bone. In any preceding or subsequent example, contact of surgical instrument 750 with patient bone may be automatically detected and/or indicated by an operator. The bone preparation tool information (e.g., voltage) may be translated to a bone characteristic, such as bone hardness.

For example, operating data may be determined for surgical instrument 750 indicating operating characteristics for surgical instrument 750 for different bone characteristics. For instance, operating data may indicate that a burring tool requires a voltage of X to generate an operating characteristic (torque, speed of rotation, etc.) value of Y for a bone hardness of Z. If the voltage required to generate operating characteristic value of Y is greater than X (i.e., more voltage, power, etc. is required to obtain the speed of rotation for harder bone), it may be determined that the bone hardness is greater than Z, and vice versa. In another example, an operating characteristic of a burring tool may be or may include measuring a level of resistance applied as torque during a cutting operation.

In any preceding or subsequent example, computational models 844 may be trained to receive surgical instrument 750 operating data as input and to generate bone characteristic information 848 as output. For example, a computational model 844 may be trained using various operating characteristics (e.g., voltage, amperage, rotation speed, torque, power, and/or the like) for objects with known bone hardness. For instance, a burring tool computational model may be trained based on operating characteristics of the burring tool (e.g., voltage, amperage, torque, speed of rotation, resistance, and/or the like) for different types of bone, bone hardness levels, and/or the like. The burring tool computational model may be used to receive operating information for the burring tool (e.g., voltage, amperage, torque, speed of rotation, resistance, and/or the like) on a patient and provide a projected or estimated bone hardness value or range.

A measurement trial may have various shapes, sizes, and/or configurations of other physical properties. It should be appreciated that the measurement trial may be provided in any suitable shape and/or configuration, which, as will be appreciated by one of ordinary skill in the art, may be dependent on the location and type of patient's bone being fixed. For example, a measurement trial may include various bone conforming segments configured to correspond with different anatomical features. In addition, the measurement trial may be arranged and configured to span, contact, be affixed to, and/or the like various portions of a human knee, including without limitation, the tibia and/or femur.

As will be described herein, the measurement trial may include any now known or hereafter developed additional features. The implant device may be manufactured from any suitable material now known or hereafter developed, including, for example, metals, polymers, plastics, ceramics, resorbable, non-resorbable, composite materials, etc. Suitable materials may include, for example, titanium, stainless steel, cobalt chrome, polyetheretherketone (PEEK), polyethylene, ultra-high molecular weight polyethylene (UHMWPE), resorbable polylactic acid (PLA), polyglycolic acid (PGA), acetal, polyoxymethylene, combinations or alloys of such materials or any other appropriate material that has sufficient strength to be secured to and hold bone, while also having sufficient biocompatibility to be implanted into a patient's body. In any preceding or subsequent example, a bone preparation process may be used to prepare a portion of patient bony anatomy to accept a cementless (or cementless portion of a hybrid) implant based on a bone preparation plan. The cementless implant may be configured to be coupled to at least a portion of the patient bony anatomy via a friction-fit, such as an interference-fit, press-fit, and/or the like. The bone preparation process may include determining various bone characteristics of the patient bony anatomy (or portion thereof) indicative of bone quality, such as bone hardness, bone density, bone elasticity, and/or the like. A virtual bone characteristic map, grid, or other structure of the patient bony anatomy (for example, a femur or tibia and/or portions thereof for receiving an implant) may be generated that indicates bone characteristic regions (for instance, areas of bone hardness). The bone preparation process may include preparing (for instance, cutting, resecting, planing, burring, and/or the like) portions of the patient bony anatomy based on the bone preparation plan according to the bone characteristic regions. In this manner, a patient may be fitted with an implant using an interference-fit and/or other properties, such as size, shape, materials, and/or the like, that is individualized for the particular patient bone characteristics, as opposed to using standard patient population and/or manufacturer recommended information according to conventional techniques. In addition, different portions or regions of the patient bony anatomy may be prepared based on the particular bone characteristics of that region

In conventional techniques, the amount of space (i.e., the amount of interference) allocated for a friction-fit on a cementless implant has a set value, relative to the implant, designed into a cut guide. For example, after determining a cut plane that would provide a perfectly complementary fit between the implant and the bone (i.e., an “ideal” cut plane), a cut guide may be designed to create a cut plane at a pre-set offset position from the ideal cut plane, thereby providing the predetermined degree of interference fit. However, such cut guides rely on a set value, which is typically standardized across patients.

In addition, the set value must be determined ahead of creation/selection of the cut guide. Accordingly, data associated with the bone quality of the patient may not be available in order to take hardness or density into account. Furthermore, conventional processes are performed with the set value generally consistent across the bone surface for the specific patient. However, bone quality may vary from location to location even on a single bone surface of a single patient. As a result, a static offset value does not provide an ideal interference-fit over the entire bone surface

Cementless implants may have an amount of interference between the bone and the implant (i.e., so that the implant has to be forced onto the bone). An offset is made in the bone resection versus the surface of the implant. The offset forces the bone to compress against the surface of the implant during installation. Differences in bone quality in the patient can affect the malleability of the bone, and thus affect the quality of the friction-fit. Using bone preparation processes according to any preceding or subsequent examples, the offset allocated for the friction-fit may be varied along the portions of the bone depending on the quality of bone at each portion.

In any preceding or subsequent example, a bone preparation process may include evaluating bone quality, qualitatively and/or quantitatively, and determining an offset value (corresponding to an appropriate degree of interference) based on the results of the evaluation. In any preceding or subsequent example, the offset value may be applied consistently over a bone surface. In any preceding or subsequent example, different surfaces and/or bone regions may have different offset values. In one example, where bone density or hardness is low, a greater offset value may be required to provide adequate stability. In another example, where bone density or hardness is high, a lower offset value may be required to provide adequate fit (i.e., ease of applying implant without damaging or greatly compressing the bone).

In any preceding or subsequent example, the offset value may be a measure indicating a difference in an amount of bone to retain versus a standard value to provide an adequate interference-fit between the bone and the implant. In an alternative example, the offset value may be a measure indicating a difference in an amount of bone to remove versus a standard value to provide an adequate interference-fit between the bone and the implant. For instance, using a femoral component as an example, an interference-fit of 0 mm may mean that the bone and the implant are complementary (i.e., the implant fits around the bone in contact with the bone surface, but without requiring force (or substantial force) to place the implant around the bone). However, with an interference-fit of 0 mm, the implant component is not affixed to the bone (i.e., an interference-fit is not achieved between the implant and the bone). An interference fit of 1.5 mm may indicate to retain an extra 1.5 mm of bone (compared to a complementary fit) so that the implant component must be forced over the extra 1.5 mm of bone to fit over the corresponding bone structure, thus providing compressive forces that affix the implant component to the bone (i.e., an interference-fit).

For example, manufacturer instructions for an implant may indicate an interference-fit of 1.5 mm. An offset value of 0.25 mm may be determined for a first portion of bone based on the hardness of the first portion (e.g., the bone has a lower hardness value than an acceptable or threshold hardness value for the 1.5 mm interference-fit), such that the surgeon implements a 1.75 mm interference-fit to provide more bone to facilitate adequate implant affixation.

The offset value may be expressed in various forms, including direct measurements (for instance, millimeters to retain/remove), percentages (for instance, a +/−percentage increase/decrease from the standard), and/or the like.

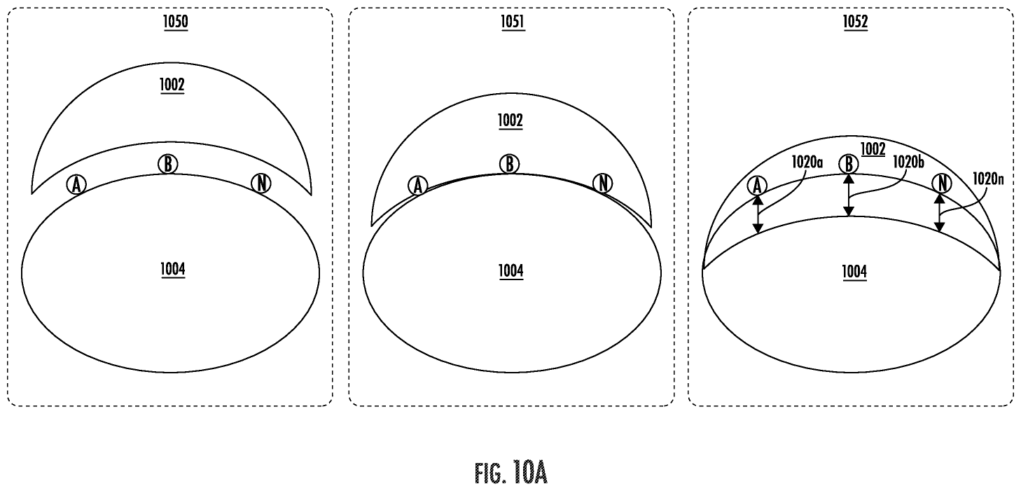

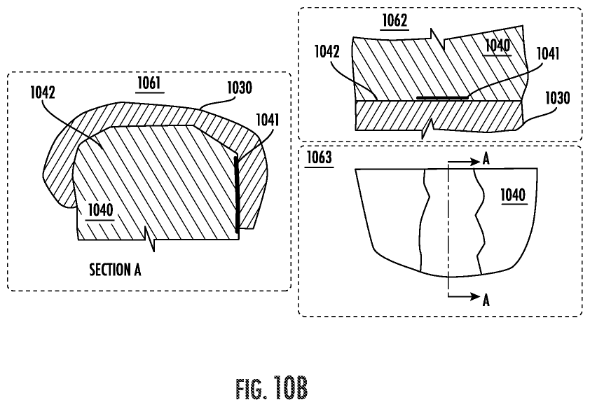

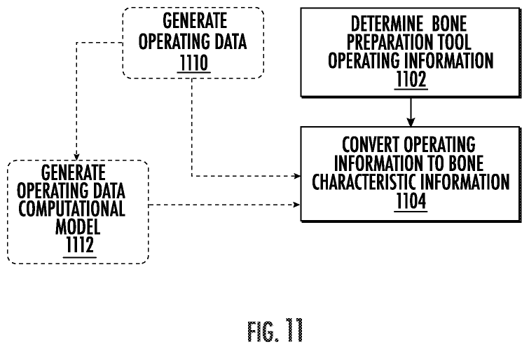

FIG. 10A illustrates a block diagram of an exemplary interference fit in accordance with one or more features of the present disclosure. As shown in FIG. 10A, an implant component 1002 may be configured to be friction fit onto a portion of a bone 1004. In a non-limiting example, implant component 1002 may be a femoral component of a TKA implant and bone portion 1004 may be a distal portion of a femur. In frame 1050, component 1002 has not yet been installed on bone portion 1004.

Component 1002 may be configured to establish a friction fit with various portions, regions, regions of interest (ROI), contact points, and/or the like. In FIG. 10A, points A-N are example contact points where component 1002 and bone portion 1004 establish (or most establish) an interference-fit. In any preceding or subsequent example, interference-fit values may be provided or determined for various contact points, such as points A-N.

In frame 1051, component 1002 has been placed on bone portion 1004 that has been prepared to be complementary to component 1002. More specifically, the outer surface dimensions of bone portion 1004 have been sized to be complementary or substantially complementary to the inner dimensions of component 1002. Accordingly, component 1002 may be placed around bone portion 1004 without requiring a material force. As a result, component 1002 is not affixed to bone portion via an interference-fit (i.e., component 1002 is loose and may be removed with minimal force).

Referring to frame 1052, bone portion 1004 has been sized with one or more interference-fit values to have an outer surface (or contact points A-N) that is larger than the corresponding inner surface (or regions that interface with contact points A-N) of component 1002. The differences 1020a-n between the size of the outer surface (or contact points A-N) of bone portion 1004 cause a force to be required to fit component 1002 over bone portion 1004, establishing the interference fit between component 1002 and bone portion 1004 to affix component 1002 to bone portion 1004.

Differences (or interference-fit values) 1020a-n may be based on various factors, such as manufacturer recommendations, surgical examination, and/or the like. Interference-fit values may have different quantities, such as about 0.1 mm, about 0.25 mm, about 0.5 mm, about 1.0 mm, about 1.5 mm, about 2.0 mm, about 3.0 mm, about 4.0 mm, about 5.0 mm, about 10 mm, and any value or range between any two of these values (including endpoints).

In any preceding or subsequent example, interference-fit values may be modified for a specific patient based on the bone quality characteristics of the patient. For example, an initial interference-fit value for area A may be 1.5 mm. However, due to a low bone hardness (for instance, a bone hardness below a threshold value), a bone preparation process according to any preceding or subsequent examples may include increasing the initial interference-fit value to provide more bone to establish an adequate fit between the implant and the bone. Accordingly, the interference-fit may be increased by a value (for instance, 0.5 mm), a percentage (for instance, 20%), and/or the like. In another example, an initial interference-fit value for point B may be 1.0 mm. However, due to a high bone hardness (for instance, a bone hardness over a threshold value), the interference-fit may be decreased by a value (for instance, 0.3 mm), a percentage, and/or the like. In another example, if normal bone has a hardness/stiffness of X kPa, and an area of the bone is 0.8X kPa and the normal interference is 0.75 mm, then the interference in that area may be modified by (modified interference)=(interference)/(hardness difference ratio) (e.g., increased to 0.75 mm/0.8 mm=0.94 mm).

FIG. 10B illustrates a block diagram of an exemplary interference fit in accordance with one or more features of the present disclosure. FIG. 10B depicts orthogonal views 1061-1063 of a femur 1040 having implant 1030 affixed on a surface thereof. In any preceding or subsequent example, femur 1040 may be prepared and/or implant 1030 may be configured to provide increased interference 1041 in areas of low bone density and/or hardness compared with areas of high bone density and/or hardness 1042.

In any preceding or subsequent example, bone characteristic information may include characteristics indicative of bone quality. In any preceding or subsequent example, bone characteristic information may include bone hardness. In any preceding or subsequent example, bone characteristic information may include bone density. In any preceding or subsequent example, the bone characteristic information may include or may be expressed using virtual maps or other graphical user interface structures that may be presented, for instance, via display device 730. Non-limiting examples of bone characteristic maps may include a hardness map (for instance, model 905 of FIG. 9A), a hardness region indicator, and/or the like. FIG. 9B illustrates an exemplary bone region indicator in accordance with one or more features of the present disclosure. As shown in FIG. 9B, a model 920 of a femur may include different hardness regions 931 and 932.

The bone characteristic information, including bone quality, bone hardness, bone density, and/or the like, may be determined according to various processes.

For instance, in any preceding or subsequent example, one or more bone characteristics may be determined via physical examination. For example, a surgeon may provide input to bone quality through sight, touch, or other physical evaluation. The surgeon may have a sense for the bone quality based on a physical (for instance, touching, pushing, etc. on the bone) and/or visual evaluation and can adjust the degree of interference up or down prior to resection based on their expertise. A physical evaluation may be performed intra-operatively.

In another example, one or more bone characteristics may be determined using a measurement trial according to any preceding or subsequent examples.

In any preceding or subsequent example, bone characteristic information may be determined based on biometric information that may be used to estimate bone quality. For example, bone quality may exhibit trends with respect to certain biometric information. Accordingly, biometric information may be used as a metric of bone quality. In one example, gender and age are highly correlated with bone quality, so this biometric information may be used to adjust the degree of interference up or down. Additionally, activity level or fitness level may be used as biometric bone quality indicators. Such biometric information can be considered by the surgeon to adjust the degree or interference based on their expertise and/or the biometric information may be input to a CASS or other planning system to adjust the degree of interference based on the factors in conjunction with an establish formula, clinical data sets, machine learning, and/or the like. The biometric information may be collected or evaluated pre-operatively and/or intra-operatively to determine an appropriate degree of interference.

Other methods for determining bone characteristic information may include pre-operative diagnostic imaging, such as CT, dual-energy X-ray absorptiometry (DEXA), and/or the like. For example, DEXA is a non-invasive test that measures bone mineral density to assess if a person is at risk of osteoporosis or fracture. FIG. 9C illustrates exemplary bone characteristic information determined via DEXA in accordance with one or more features of the present disclosure. More specifically, FIG. 9C shows bone density information determined from a patient DEXA scan. Information from a DEXA scan may facilitate regionally varying the offset to account for bone quality differences across the surface of the bone.

In any preceding or subsequent example, bone preparation tool information may be used to determine one or more bone characteristics. For example, bone tools, such as burrs, saws, and/or the like may operate differently based on engagement with bone of different characteristics. For example, the amount of power (resistance, voltage, amperage, torque, and/or other operating characteristics) required for a burr to remove bone from a bone surface may vary according to bone hardness (or another bone quality characteristic) (e.g., a voltage of X indicates a bone hardness of Y; a voltage of T indicates a bone hardness of U; and so on). In another example, the amperage (or other operating characteristic) required for a bone saw to maintain a speed (revolutions per minute and/or the like) may vary depending on the hardness of the bone engaging the saw blade.

Accordingly, in any preceding or subsequent example, operating characteristics of bone preparation tools may be monitored and used to determine bone characteristics, such as bone hardness. In any preceding or subsequent example, a CASS or other system may include operating information for various bone preparation tools that connects operating characteristics with bone quality indicators. For instance, a computational model (ML, AI, and/or the like), database, table, or other data structure may be configured that provides a bone hardness value (or range) output based on a bone preparation tool operating characteristic input. For example, a burr amperage may be provided to a ML model and the ML model may determine a bone hardness value for the bone surface being engaged by the burr tool.

In any preceding or subsequent example, a robotic bone preparation tool, such as a robotic burr tool, may be used to provide tailored cut surfaces that adapt the degree of interference based on the quality (i.e., hardness or density) of the bone. The quality of the bone may be evaluated, and the cut surface may be adapted based on the quality to achieve a patient-specific interference-fit that provides a suitable degree of interference in real-time or substantially real-time.

In one example, the quality of the bone may be evaluated prior to cutting by applying a robotic burr locally to the bone and measuring the level of resistance applied as torque to the burr during a cutting operation. The adapted cut may then be performed with the aid of robotic guidance, for instance, with a guided burr (i.e., instead of using a cut guide). Accordingly, the entire process of evaluating bone quality and completing the resection may be performed intraoperatively. This technique not only facilitates patient-specific offsets rather than a static pre-set value, but also provides the most relevant bone quality data for the cut (i.e., patient specific, in real time, and evaluated local to the cut surface).

For instance, the voltage required to operate a burr tool (or other bone preparation tool) at different levels of bone hardness may be determined and provided as pre-set values within a CASS. During a bone preparation process, the voltage of the burr tool may be determined by the CASS and compared with the pre-set values to determine a bone hardness value. The hardness value may then be used to determine an interference-fit value (for instance, based on a predefined interference-fit value modified based on the bone hardness).

In one example, the bone preparation tool (for instance, a robotic burr, CORI handpiece tool, and/or the like) may be used to collect force/displacement information (for instance, to generate a force/displacement curve) at various locations by measuring the exposure motor current vs displacement while the operator holds the tool on the bone. In general, the burr may operate as an indenter in this example. In any preceding or subsequent example, the force/displacement characteristics may be calibrated based on the configuration of the bone preparation tool (for instance, flat versus round ended burr).

Accordingly, in any preceding or subsequent example a robotic burr or other bone preparation tool may be used intra-operatively to determine bone hardness. Such examples may facilitate regionally varying the offset to account for bone quality differences across the surface.

For example, the burr could perform this function during a resection by measuring the level of resistance (torque) to the burr and adjusting the depth of the resection in near real-time. For example, the surgical plan may include a planned cut path for the robotic burr that incorporates a predetermined offset value as a default. As the burr is applied to cut the surface, the burr senses the torque, and the CASS determines the bone quality based on the torque. In real-time, the planned cut path may be adjusted to increase or decrease the degree of interference based on the determination of bone hardness.

In any preceding or subsequent example, bone analysis preparations may be performed prior to the actual bone preparation for accepting the implant. The analysis bone preparations may include cuts, resections, burring, and/or the like for analysis purposes to determine bone quality for a final resection or other preparation.

For example, in any preceding or subsequent example, adjustments may be determined across the surface immediately prior to performing a final resection. For example, in a “first pass” process, a first pass of the resection may be performed with burr across the surface at a somewhat shallower depth than the pre-planned final resection surface depth. The torque (or other operating characteristic) on the burr is measured throughout the first pass and used to determine bone quality, which then provides a map of bone quality across the entire surface, where the bone quality at any given point is determined by a local data point (i.e., the torque at a corresponding location but at the shallower depth). Based on this information, a final interference map is determined to guide a second pass with the burr to achieve the final resection surface. At some points along the second pass, additional bone may be removed (or, conversely, retained) based on the determine bone hardness.

In another example, a pilot hole process may be used to determine bone characteristics. FIG. 9D illustrates an exemplary bone analysis preparation in accordance with one or more features of the present disclosure. As shown in FIG. 9D, one or more pilot holes 951 may be prepared across the resection surface with the holes at a depth at or shallower than a possible final surface depth. For example, pilot holes 951 may be evenly spaced to define regions and/or unevenly spaced (e.g., corresponding to anatomical regions). For example, some regions may anatomically have greater or lesser bone density/hardness. The torque (or other operating characteristic) on the burr, drilling tool, and/or the like could be measured at each of the one or more pilot holes 302 providing a map 942 of bone quality 301 to guide a second pass achieving the final surface depth.

FIG. 11 illustrates an example of a method flow 1100. Method flow 1100 may be representative of some or all of the operations of determining bone characteristic information of a patient using a measurement bone preparation tool according to any preceding or subsequent examples.

Method flow 1100 may include determining operating information for a measurement bone preparation tool at block 1102. For example, during a bone preparation step of a surgical procedure (or during a pre-operative, exploratory procedure) a measurement bone preparation tool may be used to prepare a portion of patient bone, such as burring, cutting, and/or the like a portion of a tibia and/or femur. Operating information of the measurement bone preparation tool may be determined, such as via one or more sensors configured to measure voltage, amperage, torque, rotation speed (for instance, of a burr, saw blade, bit, and/or the like). The operating information may be provided to a computing device, such as a CAS (see, for instance, FIGS. 7 and 8).

At block 1104, method flow 1100 may include converting the operating information to bone characteristic information. For example, computing device 810 may use an operating data database, computational model, look-up table, and/or the like to translate one or more operating characteristics of a measurement bone preparation tool into a bone characteristic value. For instance, the operating characteristics may include a voltage or a voltage and a corresponding torque and/or rotation speed (e.g., how many volts are required to operate a burr or saw blade at a certain speed or torque). This operating information may be provided to a database, computational model, look-up table, and/or the like to convert the voltage, voltage/torque, voltage/speed, etc. to a corresponding bone hardness.

In any preceding or subsequent example, the conversion of operating information to bone characteristic information may include the use of other or additional data, such as the depth of the tool within the bone, location of the tool, type of bone, patient characteristics (e.g., age, gender, and/or the like), tool configuration (e.g., type of blade, bur, and/or the like; a condition of the tool, such as the operating age or condition of the blade, bur, and/or the like). For instance, a voltage/torque value of V may indicate a hardness of H at a depth within the bone of Y, but the voltage/torque value of V may indicate a hardness of T at a depth greater than Y.

In any preceding or subsequent example, method flow 1100 may include generating operating data at optional block 1110. For example, a manufacturer, research facility, healthcare provider, and/or the like may determine the operating characteristics of a tool for various levels of bone hardness (and/or other characteristics, such as tool depth, location, and/or the like). The operating data may be stored in a database, data file, table, and/or other data structure of file.

In any preceding or subsequent example, method flow 1100 may include generating an operating data computational model at optional block 1114. For example, a computational model may be trained using operating data of a bone preparation tool and related tool characteristics used on objects of known hardness and with known characteristics. For example, a burring tool may be used on (real or artificial) patient bone of known hardness. The operating characteristics of the burring tool may be determined during preparation of the patient bone and provided to the computational model for training, along with associated information, such as tool configuration, tool/implement operating age (e.g., burr or blade age, sharpness, size, and/or the like). In this manner, method flow 1100 may be used to generate an accurate computational model for determining bone characteristics based on the operating information of a bone preparation tool.

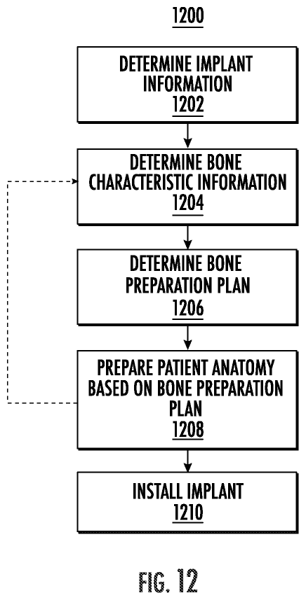

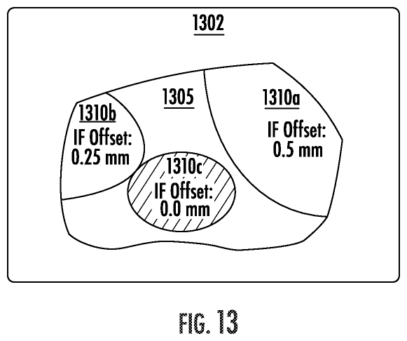

FIG. 12 illustrates an example of a method flow 1200. Method flow 1200 may be representative of some or all of the operations of a surgical procedure that includes a bone preparation process according to any preceding or subsequent examples for preparing one or more bone surfaces for installing a cementless (or cementless portion of a hybrid) implant, for example, by an operator (for example, a surgeon), a logic device (for example, a CAS system), or a combination thereof.

Method flow 1200 may include determining implant information at block 1202. For example, implant information may include dimensions, installation information, interference-fit information, interference-fit tolerances, manufacturer information, and/or the like. In any preceding or subsequent example, the implant information may include at least one interference-fit value for establishing an interference fit between at least a portion of an implant component and a corresponding portion of a bone.

At block 1204, method flow 1200 may determine bone (or bone quality) characteristic information. For example, the bone characteristic information may include bone hardness information determined via physical inspection, a measurement trial, biometric information, diagnostic imaging, bone preparation tool information, bone analysis preparations, combinations thereof, and/or the like. Determination of bone characteristics may be determined pre- and/or intra-operatively.

In any preceding or subsequent example, sensors, including, without limitation, force sensors, pressure sensors, torque sensors, displacement transducers, and/or the like, may be configured to determine force-displacement. Accordingly, in any preceding or subsequent example, data may be captured of force displacement over a known area of the bone, including, for instance, a force-displacement curve. The force-displacement information may be used to determine bone characteristics, such as bone hardness

Multiple methods for determining bone characteristics according to any preceding or subsequent examples may be used in combination. For example, imaging (e.g., DEXA) could be used to set the initial depth of a first pass or pilot hole process. In another example, a scan could be used as a check on real-time operation, for instance, warning the surgeon if the burr detected bone quality that is outside of a threshold from the imaging value.

Method flow 1200 may determine a bone preparation plan at block 1206. For example, a bone preparation plan that includes interference-fit values for one or more implant component contact points may be generated automatically by a CASS. In any preceding or subsequent example, the interference-fit values may be based on the bone characteristic information, such as bone hardness. The bone preparation plan may include preparing (for instance, burring, cutting, resecting, and/or the like) portions of the bone to achieve an interference-fit value adjusted based on bone hardness. In any preceding or subsequent example, determining, developing, generating, and/or otherwise interacting with a bone preparation plan may include updating, revising, or otherwise modifying an existing bone preparation plan.

In any preceding or subsequent example, the bone preparation plan may include an interference-fit value map or other visual structure configured to present interference-fit regions. FIG. 13 illustrates an exemplary interference-fit map in accordance with one or more features of the present disclosure. As shown in FIG. 13, an interference-fit map 1302 may present different interference-fit regions 1310a-c and their interference-fit information, such as offset values (for instance, offset values from standard), interference-fit values (for instance, the amount of bone to remove), and/or the like. In any preceding or subsequent example, interference-fit map 1302 may be presented via a CASS and used by the surgeon to prepare the patient anatomy to receive the implant. In any preceding or subsequent example, interference-fit map 1302 may be a data structure used to control a robotic bone preparation tool (such as a burr, bone saw, and/or the like) to automatically control the bone preparation tool to prepare a bone structure according to interference-fit map 1302. In any preceding or subsequent example, the interference-fit values may be used to determine a patient-specific cut guide that may be configured or fabricated for the patient. The patient-specific cut guide may be used to prepare a bone surface using bone preparation tools according to any preceding or subsequent examples.

In any preceding or subsequent example, determination of a bone preparation plan may include adaptation based on the actual dimensions of the implant. For example, rather than relying on a manufacturing tolerance to determine the fit, the actual implant dimensions could be used to customize the bone preparation plan. This would relieve some of the manufacturing burden (wider tolerances) and may facilitate determining the actual press fit on the bone. For example, the actual implant dimensions could be reported as a part of the manufacturing process, determined in the operating room before implantation, and/or the like. With the implant information, the bone preparation plan could be adjusted to accommodate and achieve the preferred interference regardless of implant dimensions, therefore removing manufacturing tolerances from the final fit.

Method flow 1200 may include preparing the patient anatomy based on the bone preparation plan at block 1208. In any preceding or subsequent example, an optional method step may include re-evaluating the bone characteristic information after the preparation, for example, to see if the bone preparation exposed bone with different bone quality characteristics. In any preceding or subsequent example, the bone preparation plan may be adjusted based on the re-evaluation of the resected bone surfaces.

At block 1210, method flow 1200 may include installing the implant. For example, a cementless TKA implant may be affixed to the knee structures using an interference-fit determined according to any preceding or subsequent examples.

In any preceding or subsequent example, determining a bone preparation plan at block 1206 may include modifying a prior or existing bone preparation plan. In some examples, the interference and/or offsets (see, for instance, FIG. 13) may be modified while other plan elements remain consistent, such as location, orientation, and/or the like. In some examples, modification of a bone preparation plan may occur at a global or local level, for instance, depending on the bone preparation step, tool, and/or the like. For example, a global modification of an interference or offset may use an average hardness for a surface, for instance, when preparing an entire surface using a saw, reamer, and/or the like. In another example, local modifications based on local offsets may be used for more precise preparation, for instance, using a burring tool. In some example, the bone preparation plan may include a tool size (e.g., saw blade, burr head, and/or the like) to achieve an appropriate level of interference, offset, and/or the like.

In any preceding or subsequent example, a measurement trial and/or measurement bone preparation tool may be used to determine hoop stresses for acetabular cup implants and stems. For example, a surgeon may ream the acetabulum to a smaller than final size (e.g., block 1204), insert a measurement device (e.g., a measurement trial configured according to some examples), to determine a hardness of the reamed hemisphere (e.g., block 1204). Then, based on this information a final reaming may be recommended, for example, in a new or updated bone preparation plan (e.g., block 1208).

The foregoing description has broad application. Accordingly, the discussion of any embodiment or example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The term “a” or “an” entity, as used herein, refers to one or more of that entity. As such, the terms “a” (or “an”), “one or more” and “at least one” can be used interchangeably herein. The use of “including,” “comprising,” or “having” and variations thereof herein is meant to encompass the items listed thereafter and equivalents thereof as well as additional items. Accordingly, the terms “including,” “comprising,” or “having” and variations thereof are open-ended expressions and can be used interchangeably herein. The phrases “at least one,” “one or more,” and “and/or,” as used herein, are open-ended expressions that are both conjunctive and disjunctive in operation.

All directional references (e.g., proximal, distal, upper, underside, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order and relative sizes reflected in the drawings attached hereto may vary.

1. A computer-assisted surgical system, comprising:
a bone preparation tool; and
at least one computing device in communication with the bone preparation tool, the at least one computing device comprising:
processing circuitry; and
a memory coupled to the processing circuitry, the memory comprising instructions that, when executed by the processing circuitry, cause the processing circuitry to:
receive operating information of the bone preparation tool during contact of the bone preparation tool with a portion of patient bone, and
determine bone characteristic information of the portion of the patient bone based on the operating information;
wherein the bone characteristic information comprises bone hardness.

2. The computer-assisted surgical system of claim 1, wherein the bone preparation tool comprises at least one of a burring tool, a cutting tool, a saw, a reamer, or a drill.

3. (canceled)

4. (canceled)

5. The computer-assisted surgical system of claim 1, wherein the operating information comprises at least one of voltage, amperage, torque, resistance, or rotation speed.

6. The computer-assisted surgical system of claim 1, the memory comprising instructions that, when executed by the processing circuitry, cause the processing circuitry to:
access an interference-fit value for a cementless implant component for the portion of the patient bone, and
determine at least one implant component property of the cementless implant based on the bone characteristic information and the interference-fit value.

7. The computer-assisted surgical system of claim 6, the at least one implant component property comprising a size of the cementless implant.

8. The computer-assisted surgical system of claim 6, the at least one implant component property comprising a shape of the cementless implant.

9. The computer-assisted surgical system of claim 1, the memory comprising instructions that, when executed by the processing circuitry, cause the processing circuitry to:
access an interference-fit value for a cementless implant component for the portion of the patient bone, and
generate an offset value based on the bone characteristic information, the offset value comprising an adjustment to the interference-fit value.

10. The computer-assisted surgical system of claim 9, wherein the bone characteristic information comprises a hardness value for each of a plurality of different regions of the portion of patient bone.

11. The computer-assisted surgical system of claim 10, the memory comprising instructions that, when executed by the processing circuitry, cause the processing circuitry to generate an interference-fit value map to visually represent interference-fit values and corresponding offsets for the plurality of different regions of the portion of patient bone.

12. The computer-assisted surgical system of claim 1, the memory comprising instructions that, when executed by the processing circuitry, cause the processing circuitry to generate a bone preparation plan for a cementless implant component based, at least in part, on the bone characteristic information.

13. The computer-assisted surgical system of claim 12, the memory comprising instructions that, when executed by the processing circuitry, cause the processing circuitry to generate the bone preparation plan via updating an existing bone preparation plan based on the bone characteristic information.

14. The computer-assisted surgical system of claim 13, the memory comprising instructions that, when executed by the processing circuitry, cause the processing circuitry to update the existing bone preparation plan with a modified interference-fit value.

15. (canceled)

16. A computer-implemented method, comprising, via at least one processor of at least one computing device:
receiving operating information of a bone preparation tool during contact of the bone preparation tool with a portion of patient bone;
accessing operational data associated with the bone preparation tool with at least one object of known bone characteristic information; and
converting the operating information to patient bone characteristic information for the portion of patient bone based on the operational data;
wherein the bone characteristic information comprises bone hardness.

17. The computer-implemented method of claim 16, wherein the bone preparation tool comprises at least one of a burring tool, a cutting tool, a saw, a reamer, or a drill.

18. (canceled)

19. (canceled)

20. The computer-implemented method of claim 16, wherein the operating information comprises at least one of voltage, amperage, torque, resistance, or rotation speed.

21. The computer-implemented method of claim 16, further comprising:
accessing an interference-fit value for a cementless implant component for the portion of the patient bone, and
determining at least one implant component property of the cementless implant based on the bone characteristic information and the interference-fit value.

22. The computer-implemented method of claim 21, the at least one implant component property comprising a size of the cementless implant.

23. The computer-implemented method of claim 21, the at least one implant component property comprising a shape of the cementless implant.

24. The computer-implemented method of claim 16, further comprising:
accessing an interference-fit value for a cementless implant component for the portion of the patient bone, and
generating an offset value based on the bone characteristic information, the offset value comprising an adjustment to the interference-fit value.

25. The computer-implemented method of claim 24, wherein the bone characteristic information comprises a hardness value for each of a plurality of different regions of the portion of patient bone.

26. The computer-implemented method of claim 25, further comprising generating an interference-fit value map to visually represent interference-fit values and corresponding offsets for plurality of different regions of the portion of patient bone.

27. The computer-implemented method of claim 25, further comprising generating a bone preparation plan for a cementless implant component based on the bone characteristic information.

28. The computer-implemented method of claim 27, further comprising generating the bone preparation plan via updating an existing bone preparation plan based on the bone characteristic information.

29. The computer-assisted surgical system of claim 28, the memory comprising instructions that, when executed by the processing circuitry, cause the processing circuitry to update the existing bone preparation plan with a modified interference-fit value.

30. (canceled)

31-60. (canceled)
